(19) United States
(12) Patent Application Publication
(10) Pub. No.: US 2025/0099177 A1
(43) Pub. Date: Mar. 27, 2025

(54) ROBOT-ASSISTED LASER OSTEOTOMY

(71) Applicant: Australian Institute of Robotic Orthopaedics PTY LTD, Crawley (AU)

(72) Inventors: Riaz Jan Kjell Khan, Cottesloe (AU); Daniel Paul Fick, Cottesloe (AU); William Brett Robertson, North Fremantle (AU); Richard Chipper

(73) Assignee: Australian Institute of Robotic Orthopaedics PTY LTD, Crawley, WA (AU)

(21) Appl. No.: 18/576,956

(22) PCT Filed: Jul. 8, 2022
(86) PCT No.: PCT/AU2022/050714

§ 371 Date: Jan. 5, 2024

(30) Foreign Application Priority Data
Jul. 8, 2021 (AU) 2021902093

(51) Int. Cl. A61B 18/20; A61B 18/00; A61B 34/10; G06T 7/00; G16H 30/40

(52) U.S. Cl. CPC A61B 18/20; G06T 7/0012; G16H 30/40; A61B 2018/00577; A61B 2034/105; G06T 2207/10088

(57) ABSTRACT

Systems, methods, and storage media for robot-assisted laser osteotomy are disclosed. Exemplary implementations may: receive osteotomy data obtained in connection with robot-assisted laser osteotomy; analyse the osteotomy data to determine one or more attributes associated with osteotomy; and determine, based on the one or more attributes, one or more functions associated with osteotomy.

### TECHNICAL FIELD

The present disclosure relates generally to analysis of bone and soft tissue. More specifically, the disclosure relates to osteotomy within an intraoperative environment.

The invention has been developed primarily for use in methods and systems for and relating to orthopaedic surgery and, in particular to systems and methods for robot-assisted laser osteotomy in orthopaedic surgery applications including post-operative care and evaluation of orthopaedic surgical procedures and will be described hereinafter with reference to this application. However, it will be appreciated that the invention is not limited to this particular field of use.

### BACKGROUND

Any discussion of the background art throughout the specification should in no way be considered as an admission that such background art is prior art, nor that such background art is widely known or forms part of the common general knowledge in the field in Australia or worldwide.

All references, including any patents or patent applications, cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art, in Australia or in any other country.

Understanding the state and classification of tissue allows various changes and precautions to be taken during surgery which can increase patient success and recovery rates. This is especially apparent in surgeries involving the musculoskeletal system where poor tissue state can have physical implications on body movement and range, likely accompanied by a degree of pain. Articular tissue such as cartilage, muscle and bone comprise the joints within this system that allow it to function, with its performance naturally degrading as they do.

Total knee arthroplasty is a prominent form of orthopedic surgery where a predefined amount of hard tissue must be removed from bones participating in the knee joint using an osteotomy. Prosthetic implants are then fixated to the remaining bone to replace that which was removed. This surgery is typically required when the cartilage surrounding the femur, tibia and patella bones start to dissipate. This causes them to grind against each other during normal movement and withstand increased levels of stress that would normally have been absorbed by the cartilage. By inserting a prosthetic implant into these bones, which is designed to absorb stress in place of them, these effects can be significantly reduced.

### SUMMARY

One aspect of the present disclosure relates to a system configured for robot-assisted laser osteotomy. The system may include one or more hardware processors configured by machine-readable instructions. The processor(s) may be configured to receive osteotomy data obtained in connection with robot-assisted laser osteotomy. The osteotomy data may include one or more of device data, medical data, and/or outcome data. The processor(s) may be configured to analyse the osteotomy data to determine one or more attributes associated with osteotomy. The processor(s) may be configured to determine, based on the one or more attributes, one or more functions associated with osteotomy. The one or more functions may include one or both of an intraoperative function or a post-operative function.

In some implementations of the system, the processor(s) may be configured to transmit, via a network, to a remote computing platform, one or more instructions to perform the one or more functions. In some implementations of the system, the one or more functions may be interpreted and carried out by a hardware processor on the remote computing platform.

In some implementations of the system, the device data may include one or more of video data, laser data, operating data, hyperspectral data, and/or orthosensor data. Alternatively, the device data may include one or more of video data, laser data, operating data, hyperspectral data, and/or joint kinematics.

In some implementations of the system, soft tissue damage may be detected and/or measured based on the hyperspectral data. Alternatively, the laser data may include one or more of laser pulse energy, laser frequency, laser fluence, water delivery and/or ablation patterns.

In some implementations of the system, the device data (or elements or combinations thereof) may be compiled and represented as a digital asset on a distributed/decentralized ledger system (e.g., blockchain). Further, the decentralized ledger may store, for example, digital asset contractual details, access rights, and/or ownership rights.

In some implementations of the system, the digital asset may comprise a unique asset or a non-fungible token (NFT). The digital asset may be managed, transferred, or involved in a smart contract.

In some implementations of the system, bone quality may be determined based on the hyperspectral data. Alternatively, the joint kinematics data may be obtained via a force and/or strain sensor.

In some implementations of the system, the orthosensor data may be obtained via an orthosensor which, in functionality, may be capable of sensing soft tissue loading and/or balancing. In some implementations of the system, the orthosensor may include a poly insert.

In some implementations of the system, the medical data may include one or more of narrative textual data, numerical measurements, recorded signals, images, pain scores, and/or demographics. Alternatively, the medical data may include one or more of narrative textual data, pre-operative medical scans, numerical measurements, recorded signals, images, pain scores, and/or demographics.

In some implementations of the system, the outcome data may include one or more of subject outcome, survivorship, scans, bone surface data before osteotomy, and/or bone surface data after osteotomy. Alternatively, the outcome data may include one or more of subject outcome, survivorship, post-operative medical scans, bone surface data before osteotomy, and/or bone surface data after osteotomy.

In some implementations of the system, the osteotomy data may be associated with a specific robot-assisted laser osteotomy operation performed on a specific subject.

In some implementations of the system, the osteotomy data may be aggregated from among a plurality of robot-assisted laser osteotomy operations performed on a plurality of subjects. Alternatively, one or more attributes may include one or more of a bone quality, a cut quality, a digital twin, and/or a laser pulse ablation measurement.

In some implementations of the system, the one or more attributes may include one or more of a cut quality, a tissue analysis, a hyperspectral result, and/or a laser pulse ablation measurement.

In some implementations of the system, the cut quality may relate to one or more of kerf width, striation patterns, and/or cutting geometry. Alternatively, the cut quality may relate to one or more of surface roughness, cut precision and/or cutting geometry

In some implementations of the system, the bone quality may relate to the mechanical and biological properties of bone. This may include, but is not limited to, bone density, bone strength, cell health and bone composition. In a further alternative embodiment, the bone quality may be determined from device data and/or medical data.

In some implementations of the system, the tissue analysis may include a hyperspectral analysis of tissue.

In some implementations of the system, the tissue analysis may be provided as a render.

In some implementations of the system, the tissue analysis may be used as an input for machine learning.

In some implementations of the system, the digital twin may include one or more of a 3D representation of the joint, tissue differentiation of the biological structures, location of the cut surfaces and/or location of the robotic system. Further, the digital twin may be determined based on device data and medical data. Still further, the digital twin may be provided as a render. Still further, the digital twin may be used as an input for machine learning and/or may be provided to a surgeon during osteotomy.

In some implementations of the system, the tissue analysis may be provided to a surgeon during osteotomy.

In some implementations of the system, differences in laser pulse ablation between subjects may be determined based on the one or more attributes.

In some implementations of the system, the differences in laser pulse ablation between subjects may include the differences between laser pulses required to ablate a given amount of tissue.

In some implementations of the system, the differences in laser pulse ablation between subjects may be a basis for determining one or both of bone quality and/or time to completion.

In some implementations of the system, the differences in laser pulse ablation between subjects may be a basis for determining a safe set of parameters that is safe, fast, and clean for an individual subject.

In some implementations of the system, the differences in laser pulse ablation between subjects may be a basis for determining a customized laser dose based on a given subjects own body and conditions.

In some implementations of the system, data-informed implant designs may be designed based on the one or more attributes.

In some implementations of the system, the data-informed implant designs may be tailored for individual subjects.

In some implementations of the system, the data-informed implant designs may include a set of standard implant sizes.

In some implementations of the system, the one or more attributes may be associated with a specific robot-assisted laser osteotomy operation performed on a specific subject.

In some implementations of the system, the one or more attributes may be aggregated from among a plurality of robot-assisted laser osteotomy operations performed on a plurality of subjects.

In some implementations of the system, the intraoperative function may include one or more of a fully autonomous surgery function, an implant positioning guidance function, a point of no return warnings function, and/or a time to completion estimates function.

In some implementations of the system, the fully autonomous surgery function may facilitate a trained machine learning system in autonomously performing a procedure.

In some implementations of the system, the implant positioning guidance function may facilitate determining an optimal position of an implant.

In some implementations of the system, determining an optimal position of an implant may include dynamically aligning a joint using a digital twin of the joint under different scenarios.

In some implementations of the system, the digital twin of the joint may be determined based on a functional magnetic resonance imaging scan.

In some implementations of the system, the soft tissue damage detection function may facilitate notification of a surgeon to potential damage to soft tissue structures throughout the osteotomy process.

In some implementations of the system, the point of no return warnings may function facilitates provisioning of a warning to a surgeon as a point of no return in bone ablation is approaching and/or has passed.

In some implementations of the system, the time to completion may estimate function facilitates providing a surgeon with a time estimate for how much longer a tissue resection process and/or osteotomy process will take.

In some implementations of the system, the post-operative function may include one or more of an operation outcome reports function, a data-informed rehabilitation plans function, a surgeon certification function, a certification via verifiable use statistics function, and/or a collated results function.

In some implementations of the system, the operation outcome may report function facilitates generation of an operation outcome report with intra-operatively collected data.

In some implementations of the system, the operation outcome report may include one or more of data associated with bone tissue before osteotomy and bone tissue after osteotomy, implant-bone fit, cut quality, soft-tissue damage, joint balancing, spectral analysis of bone, and/or bone quality.

In some implementations of the system, the operation outcome report may be used as an input for fully autonomous surgery and/or as a training input for fully autonomous surgery.

In some implementations of the system, the data-informed rehabilitation may plan function facilitates providing recommendations regarding rehabilitation of a given subject based on collected intraoperative and post-operative information.

In some implementations of the system, the certification via verifiable use statistics may function facilitates issuance of a certification provided to surgeons after completing a certain number of procedures. Alternatively, the surgeon certification function may facilitate issuance of a certification provided to surgeons after completing a certain number of procedures.

In some implementations of the system, the collated results function may facilitate collating osteotomy data.

Another aspect of the present disclosure relates to a method for robot-assisted laser osteotomy. The method may include receiving osteotomy data obtained in connection with robot-assisted laser osteotomy. The osteotomy data may include one or more of device data, medical data, and/or outcome data. The method may include analysing the osteotomy data to determine one or more attributes associated with osteotomy. The method may include determining, based on the one or more attributes, one or more functions associated with osteotomy. The one or more functions may include one or both of an intraoperative function or a post-operative function.

In some implementations of the method, it may further include transmitting, via a network, to a remote computing platform, one or more instructions to perform the one or more functions. In some implementations of the method, the one or more functions may be interpreted and carried out by a hardware processor on the remote computing platform.

In some implementations of the method, the device data may include one or more of video data, laser data, operating data, hyperspectral data, joint kinematics data and/or orthosensor data. In some implementations of the method, the device data includes one or more of video data, laser data, operating data, hyperspectral data, and/or joint kinematics data.

In some implementations of the method, the laser data may include one or more of laser pulse energy, laser frequency, laser fluence, water delivery and/or ablation patterns.

In some implementations of the method, the joint kinematics data may be obtained via a force and/or strain sensor.

In some implementations of the method, the medical data may include one or more of narrative textual data, pre-operative medical scans, numerical measurements, recorded signals, images, pain scores, and/or demographics.

In some implementations of the method, the outcome data may include one or more of subject outcome, survivorship, post-operative medical scans, bone surface data before osteotomy, and/or bone surface data after osteotomy.

In some implementations of the method, the osteotomy data may be associated with a specific robot-assisted laser osteotomy operation performed on a specific subject.

In some implementations of the method, the osteotomy data may be aggregated from among a plurality of robot-assisted laser osteotomy operations performed on a plurality of subjects.

In some implementations of the method, one or more attributes may include one or more of a bone quality, a cut quality, a digital twin, and/or a laser pulse ablation measurement.

In some implementations of the method, the bone quality may relate to the mechanical and biological properties of bone. This may include, but is not limited to, bone density, bone strength, cell health and bone composition.

In some implementations of the method, the bone quality may be determined from device data and/or medical data.

In some implementations of the method, the cut quality may relate to one or more of surface roughness, cut precision and/or cutting geometry.

In some implementations of the method, the digital twin may include one or more of a 3D representation of the joint, tissue differentiation of the biological structures, location of the cut surfaces and/or location of the robotic system.

In some implementations of the method, the digital twin may be determined based on device data and medical data.

In some implementations of the method, the digital twin may be provided as a render.

In some implementations of the method, the digital twin may be used as an input for machine learning, and/or may be is provided to a surgeon during osteotomy.

In some implementations of the method, differences in laser pulse ablation between subjects may be determined based on the one or more attributes.

In some implementations of the method, the differences in laser pulse ablation between subjects may include the differences between laser pulses required to ablate a given amount of tissue.

In some implementations of the method, the differences in laser pulse ablation between subjects may be a basis for determining one or both of bone quality and/or time to completion.

In some implementations of the method, the differences in laser pulse ablation between subjects may be a basis for determining a safe set of parameters that is safe, fast, and clean for an individual subject.

In some implementations of the method, the differences in laser pulse ablation between subjects may be a basis for determining a customized laser parameters based on a given subjects own body and conditions.

In some implementations of the method, data-informed implant designs may be designed based on the one or more attributes.

In some implementations of the method, the data-informed implant designs may be tailored for individual subjects.

In some implementations of the method, the data-informed implant designs may include a set of standard implant sizes.

In some implementations of the method, the one or more attributes may be associated with a specific robot-assisted laser osteotomy operation performed on a specific subject.

In some implementations of the method, the intraoperative function may include one or more of a fully autonomous surgery function, an implant positioning guidance function, soft tissue damage detection function, a point of no return warning function, and/or a time to completion estimate function.

In some implementations of the method, the fully autonomous surgery function may facilitate a trained machine learning system in autonomously performing a procedure.

In some implementations of the method, the implant positioning guidance function may facilitate determining an optimal position of an implant.

In some implementations of the method, determining an optimal position of an implant may include dynamically aligning a joint using a digital twin of the joint under different scenarios.

In some implementations of the method, the soft tissue damage detection function may facilitate notification of a surgeon to potential damage to soft tissue structures throughout the osteotomy process.

In some implementations of the method, the point of no return warning function may facilitate provisioning of a warning to a surgeon as a point of no return in bone ablation is approaching and/or has passed.

In some implementations of the method, the time to completion estimates function may facilitate providing a surgeon with a time estimate for how much longer an osteotomy process will take.

In some implementations of the method, the post-operative function may include one or more of an operation outcome report function, a data-informed rehabilitation plan function, a surgeon certification function, and/or a collated results function.

In some implementations of the method, the operation outcome report function may facilitate generation of an operation outcome report from intra-operatively collected data.

In some implementations of the method, the operation outcome report may include one or more of data associated with bone tissue before osteotomy and bone tissue after osteotomy, implant-bone fit, cut quality, soft-tissue damage, joint balancing, and/or bone quality.

In some implementations of the method, the operation outcome report may be used as a training input for fully autonomous surgery.

In some implementations of the method, the data-informed rehabilitation plan function may facilitate providing recommendations regarding rehabilitation of a given subject based on collected intra-operative and post-operative information.

In some implementations of the method, the surgeon certification function may facilitate issuance of a certification provided to surgeons after completing a certain number of procedures.

In some implementations of the method, the collated results function may facilitate collating osteotomy data.

In some implementations of the method, soft tissue damage may be detected and/or measured based on the hyperspectral data.

In some implementations of the method, bone quality may be determined based on the hyperspectral data.

In some implementations of the method, the orthosensor data may be obtained via an orthosensor which, in functionality, may be capable of sensing soft tissue loading and/or balancing. In some implementations of the method, the orthosensor may include a poly insert.

In some implementations of the method, the medical data may include one or more of narrative textual data, numerical measurements, recorded signals, images, pain scores, and/or demographics.

In some implementations of the method, the outcome data may include one or more of subject outcome, survivorship, scans, bone surface data before osteotomy, and/or bone surface data after osteotomy.

In some implementations of the method, the osteotomy data may be associated with a specific robot-assisted laser osteotomy operation performed on a specific subject.

In some implementations of the method, the osteotomy data may be aggregated from among a plurality of robot-assisted laser osteotomy operations performed on a plurality of subjects.

In some implementations of the method, the one or more attributes may include one or more of a cut quality, a tissue analysis, a hyperspectral result, and/or a laser pulse ablation measurement.

In some implementations of the method, the cut quality may relate to one or more of kerf width, striation patterns, and/or cutting geometry.

In some implementations of the method, the tissue analysis may include a hyperspectral analysis of tissue.

In some implementations of the method, the tissue analysis may be provided as a render.

In some implementations of the method, the tissue analysis may be used as an input for machine learning.

In some implementations of the method, the tissue analysis may be provided to a surgeon during osteotomy.

In some implementations of the method, differences in laser pulse ablation between subjects may be determined based on the one or more attributes.

In some implementations of the method, the differences in laser pulse ablation between subjects may include the differences between laser pulses required to ablate a given amount of tissue.

In some implementations of the method, the differences in laser pulse ablation between subjects may be a basis for determining one or both of bone quality and/or time to completion.

In some implementations of the method, the differences in laser pulse ablation between subjects may be a basis for determining a safe set of parameters that is safe, fast, and clean for.

In some implementations of the method, the differences in laser pulse ablation between subjects may be a basis for determining a customized laser dose based on a given subjects own body and conditions.

In some implementations of the method, data-informed implant designs may be designed based on the one or more attributes.

In some implementations of the method, the data-informed implant designs may be tailored for individual subjects.

In some implementations of the method, the data-informed implant designs may include a set of standard implant sizes.

In some implementations of the method, the one or more attributes may be associated with a specific robot-assisted laser osteotomy operation performed on a specific subject.

In some implementations of the method, the one or more attributes may be aggregated from among a plurality of robot-assisted laser osteotomy operations performed on a plurality of subjects.

In some implementations of the method, the intraoperative function may include one or more of a fully autonomous surgery function, an implant positioning guidance function, a point of no return warnings function, and/or a time to completion estimates function.

In some implementations of the method, the fully autonomous surgery function may facilitate a trained machine learning system in autonomously performing a procedure.

In some implementations of the method, the implant positioning guidance function may facilitate determining an optimal position of an implant.

In some implementations of the method, determining an optimal position of an implant may include dynamically aligning a joint using a digital twin of the joint under different scenarios.

In some implementations of the method, the digital twin of the joint may be determined based on a functional magnetic resonance imaging scan.

In some implementations of the method, the point of no return warnings may function facilitates provisioning of a warning to a surgeon as a point of no return in bone ablation is approaching and/or has passed.

In some implementations of the method, the time to completion may estimate function facilitates providing a surgeon with a time estimate for how much longer a tissue resection process will take.

In some implementations of the method, the post-operative function may include one or more of an operation outcome reports function, a data-informed rehabilitation plans function, a certification via verifiable use statistics function, and/or a collated results function.

In some implementations of the method, the operation outcome may report function facilitates generation of an operation outcome report with intra-operatively collected data.

In some implementations of the method, the operation outcome report may include one or more of data associated with bone tissue before osteotomy and bone tissue after osteotomy, implant-bone fit, spectral analysis of bone, and/or bone quality.

In some implementations of the method, the operation outcome report may be used as an input for fully autonomous surgery.

In some implementations of the method, the data-informed rehabilitation may plan function facilitates providing recommendations regarding rehabilitation of a given subject based on collected intraoperative information.

In some implementations of the method, the certification via verifiable use statistics may function facilitates issuance of a certification provided to surgeons after completing a certain number of procedures.

In some implementations of the method, the collated results function may facilitate collating osteotomy data.

Yet another aspect of the present disclosure relates to a non-transient computer-readable storage medium having instructions embodied thereon, the instructions being executable by one or more processors to perform a method for robot-assisted laser osteotomy. The method may include receiving osteotomy data obtained in connection with robot-assisted laser osteotomy. The osteotomy data may include one or more of device data, medical data, and/or outcome data. The method may include analysing the osteotomy data to determine one or more attributes associated with osteotomy. The method may include determining, based on the one or more attributes, one or more functions associated with osteotomy. The one or more functions may include one or both of an intraoperative function or a post-operative function.

In some implementations of the computer-readable storage medium, the method may further include transmitting, via a network, to a remote computing platform, one or more instructions to perform the one or more functions. In some implementations of the computer-readable storage medium, the one or more functions may be interpreted and carried out by a hardware processor on the remote computing platform.

In some implementations of the computer-readable storage medium, the device data may include one or more of video data, laser data, operating data, hyperspectral data, joint kinematics data and/or orthosensor data.

In some implementations of the computer-readable storage medium, the laser data may include one or more of a laser pulse energy, laser frequency, laser fluence, water delivery and/or ablation patterns.

In some implementations of the computer-readable storage medium, the joint kinematics data may be obtained via a force and/or strain sensor.

In some implementations of the computer-readable storage medium, soft tissue damage may be detected and/or measured based on the hyperspectral data.

In some implementations of the computer-readable storage medium, bone quality may be determined based on the hyperspectral data.

In some implementations of the computer-readable storage medium, the orthosensor data may be obtained via an orthosensor which, in functionality, may be capable of sensing soft tissue loading and/or balancing. In some implementations of the computer-readable storage medium, the orthosensor may include a poly insert.

In some implementations of the computer-readable storage medium, the medical data may include one or more of narrative textual data, pre-operative medical scans, numerical measurements, recorded signals, images, pain scores, and/or demographics.

In some implementations of the computer-readable storage medium, the outcome data may include one or more of subject outcome, survivorship, post-operative medical scans, bone surface data before osteotomy, and/or bone surface data after osteotomy.

In some implementations of the computer-readable storage medium, the osteotomy data may be associated with a specific robot-assisted laser osteotomy operation performed on a specific subject.

In some implementations of the computer-readable storage medium, the osteotomy data may be aggregated from among a plurality of robot-assisted laser osteotomy operations performed on a plurality of subjects.

In some implementations of the computer-readable storage medium, the one or more attributes may include one or more of a bone quality, cut quality, a digital twin, a tissue analysis, a hyperspectral result, and/or a laser pulse ablation measurement. Further, the bone quality may relate to the mechanical and/or biological properties of the bone. This may include, but is not limited to, bone density, bone strength, cell health and/or bone composition. Still further, the bone quality may be determined from device data and/or medical data.

In some implementations of the computer-readable storage medium, the cut quality may relate to one or more of surface roughness, cut precision, kerf width, striation patterns, and/or cutting geometry.

In some implementations of the computer-readable storage medium, the digital twin may include one or more of a 3D representation of the joint, the tissue differentiation of the biological structures, the location of the cut surfaces and/or the location of the robotic system.

In some implementations of the computer-readable storage medium, the digital twin may be determined based on device data and/or medical data. Further, the digital twin may be provided as a render. Still further, the digital twin may be used as an input for machine learning. Still further, the digital twin may be provided to a surgeon during osteotomy.

In some implementations of the computer-readable storage medium, the tissue analysis may include a hyperspectral analysis of tissue.

In some implementations of the computer-readable storage medium, the tissue analysis may be provided as a render.

In some implementations of the computer-readable storage medium, the tissue analysis may be used as an input for machine learning.

In some implementations of the computer-readable storage medium, the tissue analysis may be provided to a surgeon during osteotomy.

In some implementations of the computer-readable storage medium, differences in laser pulse ablation between subjects may be determined based on the one or more attributes.

In some implementations of the computer-readable storage medium, the differences in laser pulse ablation between subjects may include the differences between laser pulses required to ablate a given amount of tissue.

In some implementations of the computer-readable storage medium, the differences in laser pulse ablation between subjects may be a basis for determining one or both of bone quality and/or time to completion.

In some implementations of the computer-readable storage medium, the differences in laser pulse ablation between subjects may be a basis for determining a safe set of parameters that is safe, fast, and clean for an individual subject.

In some implementations of the computer-readable storage medium, the differences in laser pulse ablation between subjects may be a basis for determining one or more of a set of customized laser parameters based on a given subjects own body and conditions, and a customized laser dose based on a given subjects own body and conditions.

In some implementations of the computer-readable storage medium, data-informed implant designs may be designed based on the one or more attributes.

In some implementations of the computer-readable storage medium, the data-informed implant designs may be tailored for individual subjects.

In some implementations of the computer-readable storage medium, the data-informed implant designs may include a set of standard implant sizes.

In some implementations of the computer-readable storage medium, the one or more attributes may be associated with a specific robot-assisted laser osteotomy operation performed on a specific subject.

In some implementations of the computer-readable storage medium, the one or more attributes may be aggregated from among a plurality of robot-assisted laser osteotomy operations performed on a plurality of subjects.

In some implementations of the computer-readable storage medium, the intraoperative function may include one or more of a fully autonomous surgery function, an implant positioning guidance function, a soft tissue damage detection function, a point of no return warnings function, and/or a time to completion estimates function.

In some implementations of the computer-readable storage medium, the fully autonomous surgery function may facilitate a trained machine learning system in autonomously performing a procedure.

In some implementations of the computer-readable storage medium, the implant positioning guidance function may facilitate determining an optimal position of an implant.

In some implementations of the computer-readable storage medium, determining an optimal position of an implant may include dynamically aligning a joint using a digital twin of the joint under different scenarios.

In some implementations of the computer-readable storage medium, the digital twin of the joint may be determined based on a functional magnetic resonance imaging scan.

In some implementations of the computer-readable storage medium, the soft tissue damage detection function may facilitate notification of a surgeon to potential damage to soft tissue structures throughout the osteotomy process.

In some implementations of the computer-readable storage medium, the point of no return warnings function may facilitate provisioning of a warning to a surgeon as a point of no return in bone ablation is approaching and/or has passed.

In some implementations of the computer-readable storage medium, the time to completion may estimate function facilitates providing a surgeon with a time estimate for how much longer a tissue resection process will take.

In some implementations of the computer-readable storage medium, the post-operative function may include one or more of an operation outcome reports function, a data-informed rehabilitation plans function, a surgeon certification function, a certification via verifiable use statistics function, and/or a collated results function.

In some implementations of the computer-readable storage medium, the operation outcome may report function facilitates generation of an operation outcome report with intra-operatively collected data.

In some implementations of the computer-readable storage medium, the operation outcome report may include one or more of data associated with bone tissue before osteotomy and bone tissue after osteotomy, implant-bone fit, cut quality, soft tissue damage, joint balancing, spectral analysis of bone, and/or bone quality.

In some implementations of the computer-readable storage medium, the operation outcome report may be used as an input for fully autonomous surgery and/or as a training input for fully autonomous surgery.

In some implementations of the computer-readable storage medium, the data-informed rehabilitation may plan function facilitates providing recommendations regarding rehabilitation of a given subject based on collected intraoperative and post-operative information.

In some implementations of the computer-readable storage medium, the certification via verifiable use statistics may function facilitates issuance of a certification provided to surgeons after completing a certain number of procedures. Alternatively, the surgeon certification function may facilitate issuance of a certification provided to surgeons after completing a certain number of procedures.

In some implementations of the computer-readable storage medium, the collated results function may facilitate collating osteotomy data.

### BRIEF DESCRIPTION OF THE DRAWINGS

Notwithstanding any other forms which may fall within the scope of the present disclosure, preferred embodiments will now be described, by way of example only, with reference to the accompanying drawings in which:

FIG. 1 is an abstract schematic flow diagram depicting the intraoperative tissue type and composition analysis process in accordance with an embodiment of the present disclosure;

FIG. 2 is a detailed schematic flow diagram depicting the data sources and related procedures involved in the collection and accessibility of data as introduced in the exemplary data collection step in FIG. 1;

FIG. 3 is a detailed schematic flow diagram depicting the pre-processing and manipulation used to transform data into a more evaluable form for further usage within algorithms and methods as introduced in the exemplary data processing step in FIG. 1;

FIG. 4 is a detailed schematic flow diagram depicting the types of predictive algorithms or methods capable of producing information and properties relating to tissue type and composition based on existing processed data as introduced in the exemplary data interpretation step in FIG. 1;

FIG. 5A illustrates generally a basic sensing system and a sample intraoperative environment in which it may be used;

FIG. 5B illustrates generally a hyperspectral sensing system of the system in FIG. 5A which collects spectral data based on a subject;

FIG. 5C illustrates generally an acoustic sensing system of the system in FIG. 5A which collects acoustic data based on a subject;

FIG. 6 illustrates a system configured for robot-assisted laser osteotomy in accordance with an embodiment of the present disclosure;

FIGS. 7A and/or 7B is a schematic flow diagram depicting a process for robot-assisted laser osteotomy in accordance with an embodiment of the present disclosure; and

FIG. 8 shows a computing device on which the various embodiments described herein may be implemented in accordance with an embodiment of the present disclosure.

### DEFINITIONS

The following definitions are provided as general definitions and should in no way limit the scope of the present invention to those terms alone but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (that is to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising”, “including”, “carrying”, “having”, “containing”, “involving”, “holding”, “composed of”, and the like are to be understood to be open-ended, that is to mean “including but not limited to”. Only the transitional phrases “consisting of” and “consisting essentially of” alone shall be closed or semi-closed transitional phrases, respectively.

The term, “real-time”, for example “displaying real-time data” refers to the display of the data without intentional delay, given the processing limitations of the system and the time required to accurately measure the data.

The term, “near-real-time”, for example “obtaining real-time or near-real-time data” refers to the obtaining of data either without intentional delay (“real-time”) or as close to real-time as practically possible (that is with a small, but minimal, amount of delay) whether intentional or not within the constraints and processing limitations of the system for obtaining and recording or transmitting the data.

Although any methods and materials similar or equivalent to those described herein can be used in the practise or testing of the present invention, preferred methods and materials are described. It will be appreciated that the methods, apparatus and systems described herein may be implemented in a variety of ways and for a variety of purposes. The description here is by way of example only.

As used herein, the term “exemplary” is used in the sense of providing examples, as opposed to indicating quality. That is, an “exemplary embodiment” is an embodiment provided as an example, as opposed to necessarily being an embodiment of exemplary quality and serving as a desirable model or representing the best of its kind.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a single or multiple computer readable storage mediums which may comprise computer memory, one or more floppy discs, compact discs, optical discs, magnetic tapes, flash memories, circuit configurations in Field Programmable Gate Arrays or other semiconductor devices and any other non-transitory or tangible storage media. These mediums may be encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that perform methods of the present invention when executed need not reside on a single computer or processor but may be distributed in a modular fashion amongst a number of different computers or processors to implement various aspects of the present invention.

Computer-executable instructions may exist in a variety of forms, such as program modules, and may be executed by a singular, combination or sequence of computers or other devices whose level of collaboration may differ to varying degrees. Program modules generally include routines, programs, objects, components, data structures and any other singular, combination or sequence of components, structures or executable entities that perform particular tasks, implement specific abstract data types or coordinate different processes. The functionality of a program module may typically be combined or distributed as desired in various embodiments.

Data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys the relationship between the fields. However, any suitable mechanism may be used to establish a relationship between information in fields of a data structure, including through the use of pointers, tags or other mechanisms that establish relationship between data elements.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined such that elements may be conjunctively present in some cases and disjunctively present in other cases. Multiple elements listed with “and/or” should be construed in the same fashion, that is “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); and any other embodiments which may comprise unions, intersections or otherwise other combinations of associated elements.

As used herein in the specification and in the claims, “or” should be understood to have the same meaning as “and/or” as defined above. For example, when separating items in a list, “or” or “and/or” shall be interpreted as being inclusive, that is the inclusion of at least one, but also including more than one, of a number or list of elements, and, optionally, additional unlisted items. Only terms clearly indicated to the contrary, such as “only one of” or “exactly one of”, or, when used in the claims, “consisting of” will refer to the inclusion of exactly one element of a number or list of elements. In general, the term “or” as used herein shall only be interpreted as indicating exclusive alternatives (that is “one or the other but not both”) when preceded by terms of exclusivity, such as “either”, “one of”, “only one of”, or “exactly one of”. “Consisting essentially of”, when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); and any other embodiments which may comprise differing combinations of inclusions and exclusions of A, B and any other associated elements.

For the purpose of this specification, where method steps are described in sequence, the sequence does not necessarily mean that the steps are to be carried out in chronological order in that sequence. The steps may be carried out in a completely alternate order which may mean that some occur simultaneously or are omitted entirely.

In addition, where features or aspects of the invention are described in terms of Markush groups, those skilled in the art will recognize that the invention is also thereby described in terms of any individual member or subgroup of members of the Markush group.

### DETAILED DESCRIPTION

The following detailed description is an exemplification of the invention and should not be limited in scope by the embodiments depicted nor should it be understood in any way as a restriction on the broad description of the invention as set out hereinbefore. These embodiments are described in sufficient detail to allow those skilled in the art to practise or exercise the invention. The precise shape, size and appearance of the components described or illustrated are not expected of nor required from the invention unless stated otherwise. It is to be understood that any utilisation, combination or structural, logical, electrical and mechanical changes, variations, augmentations or modifications to any of the mentioned or otherwise related embodiments may be made without departing from the scope of the invention. Similarly, any functionally equivalent products, compositions and methods will also remain within this scope along with all singular, combination and sequences of steps, features, structures, sequences, processes, combinations and compounds referred to or indicated within this description either singularly or collectively.

The entire disclosure of all documentation including patents, patent applications, journal articles, laboratory manuals, books, charts, repositories, and any other form of documentation or otherwise referenced resources cited herein is by no means an admission of prior art, prior or common knowledge required by those skilled in the art or any other connections or assumptions towards the invention unless mentioned otherwise.

Throughout this description, unless stated otherwise, the words “comprise”, “include” and any variations which may consist of “comprising”, “comprises”, “including” or “includes” will be understood to imply the inclusion of a stated integer or a group of integers but not the exclusion of any other integer or group of integers. Relative language such as “about” or “approximately” will be understood to be the application of 10% variability in the positive and negative directions of their subject unless stated or specified otherwise. Other definitions for selected terms may be found herein and will apply for the remainder of the section unless redefined otherwise. All other definitions will reflect the common understanding of those ordinarily skilled in the art of the invention or as declared by the subject area.

Features presented through the drawings are referenced using the numerical ordering of the invention stage that they belong to alongside their logical ordering within the drawing itself, with the exception of the first drawing which acts as the initial overview.

Like or the same reference numerals may be used to denote the same or similar features.

The invention will be described in terms of embodiments that relate to systems, methods, and storage media for robot-assisted laser osteotomy such as in knee operations. However, the invention has applicability more generally in the area of osteotomy.

Conventional osteotomy systems have very limited capacity or functionality for intraoperative data collection. In conventional laser osteotomy systems, data collected intraoperatively is used primarily as feedback to maintain or adjust laser and/or other operational conditions during osteotomy. Accordingly, implant designs and individual osteotomy procedures do not benefit from data of prior procedures performed using conventional laser osteotomy systems.

Implementations described herein address these and other shortcomings by collecting, aggregating and utilizing intraoperative data in combination with other relevant subject data. In some implementations, the aggregated data may facilitate fully autonomous surgery, implant positioning guidance, soft tissue damage detection, point of no return warnings, and/or time to completion estimates. In some implementations, the aggregated data may facilitate data-informed implant designs, operation outcome reports, data-informed rehabilitation plans, surgeon certification, certification via verifiable use statistics function, and/or collated results.

It should be appreciated that the invention is not limited to orthopaedic operations nor is it limited to any form or type of tissue.

Biological tissue are heterogeneous structures that can absorb, reflect, scatter and re-emit light. The light which is reflected from tissue can be detected and measured through a process called diffuse reflectance spectroscopy. The auto fluorescence light that is emitted can be detected and measured through a process called fluorescence excitation spectroscopy. The majority of tissue, including articular tissue, exhibit unique spectral characteristics in the ultraviolet, visible and near-visible ranges due to their biochemical and morphological states. By sensing and processing these unique spectra, it is possible to classify and determine the state of tissue.

This process typically involves a light source and a method of capture. The light source, whose type is dependent on the form of light being captured, must illuminate the tissue. The method of capture, such as a reflectance probe and spectrometer, will capture the result from the tissue based on this light exposure. It can be used preoperatively on tissue samples and intraoperatively on the patient without resulting in any changes to the state of the tissue. It is mechanically non-invasive as, depending on the light source used, it may be able to reach under the skin to a certain degree, although results received from this are not likely comparable to the exposed equivalent.

Biological tissue mechanically vibrates differently depending on their composition and type. When the tissue acts as the sound source through this vibration, mediums surrounding the tissue such as air and other tissue begin to vibrate as a result. This will continue as sound waves propagate away from the sound source. These sounds waves can be detected and captured through acoustic sensors such as a microphone By listening to the distinct sound, it is possible to distinguish between different tissue types and determine various aspects of their composition, such as water content or density.

This process requires an initial impact to trigger vibrations in the tissue or sound source. For example, this could be produced by a tightly controlled impact from an actuator, a laser pulse or an air jet. This will create sound waves that can be captured using an acoustic sensor which must be within range of the occurrence and have a high enough sampling rate to draw sufficient detail from. This process can be used preoperatively and intraoperatively, although care must be taken to ensure that the initial trigger impact does not cause damage to the underlying tissue. Depending on the impact trigger, it may be able to reach under the skin to a degree and provide information based on the sound produced whilst taking into account the epithelial tissue acting as a buffer. This will limit the amount of information that can be obtained, although properties such as bone density will still be readily available.

The data retrieved from these different types of sensors typically needs to be parsed, processed and transformed before it can be used in various machine learning, data science and mathematical techniques. This can involve multiple steps depending on the sensors and intended techniques involved. It begins by processing the data which is generally split into two different parts, pre-processing and manipulation. Pre-processing consists of the cleaning, rearranging, formatting and sampling of data to achieve a more usable form. Manipulation consists of scaling or aligning the pre-processed data, decomposing it into its constituent or representative elements and then aggregating the result if necessary.

This is where the training phase begins which builds up a specific machine learning, data science or mathematical technique and allows it to identify its subject. Training consists of matching a set of processed data against their tissue state or classification ground truth and determining which of their values or combination of values result in which truth. Once an adequate amount of data has been channelled through the training phase, the technique can be executed.

Utilising the specific machine learning, data science or mathematical technique once trained usually involves passing it processed data which does not have a matching tissue state or classification ground truth. The values that it contains are run through the technique and are matched against a pre-existing ground truth that was obtained through prior training. This pre-existing truth will be the predicted matching tissue state or classification of the tissue belonging to the data. This process can be repeated using data which does have a matching ground truth to determine the accuracy of a technique depending on how closely the given truth matches its actual one.

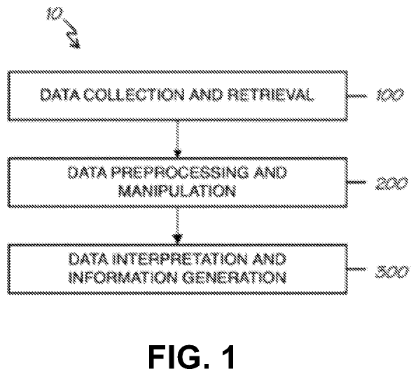

FIG. 1 is a schematic flow diagram depicting the intraoperative tissue state and class analysis method 10, segmented into the individual steps that comprise it. The flow of information between these steps and the individual processing they may contain is explained in overview. Data collection 100 utilises a series of differing sensors in possibly alternating arrangements to produce varying amounts and types of data based on a subject which may be tissue. Data processing 200 pre-processes and manipulates this data to create a form with increased usability and evaluability. Data interpretation 300 passes this processed data into various models and algorithms capable of predicting state and classification information. These models and algorithms are initially trained based on the mapping between historical analysis information and the actual corresponding results and may comprise artificial neural networks (ANNs) or the like as would be appreciated by the skilled addressee. Once populated, new processed data can be passed through wherein identified indicators will be mapped to corresponding values, predicting state and classification information.

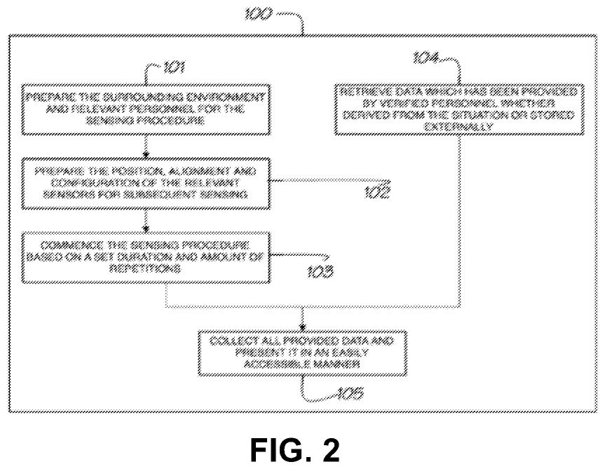

FIG. 2 is a detailed schematic diagram depicting an exemplary data collection and retrieval step 100 of the type depicted in FIG. 1. Data may be collected through a series of sensors that may differ in terms of their type, quantity and arrangement or may be retrieved through external sources related to the subject or the procedure surrounding the subject across a multitude of possible embodiments.

Sensors may act independently or cooperatively as part of a system or collective of sensors where each would cooperate in some fashion to increase the quality or amount of sensed data. Each sensor may be completely self-contained or may require additional devices or systems to handle all or some portion of the required processing.

The physical arrangement of sensors should be made to surround the subject (e.g., a joint e.g. knee, elbow, hip, shoulder etc., of a patient upon which an orthopaedic surgical procedure is being performed or has previously been performed) in a way that maximises the sensing potential of the plurality of sensors whilst resulting in the minimum amount of disturbance to the surrounding environment. If the sensors exist as part of a system in a cooperative setup, then their arrangement should reflect this, for example, sensing the subject from different angles to later stitch the different perspectives together.

The sensors may be automated, manually triggered or controlled through some combination of the two depending on the embodiment. In situations where a proper sensing environment must be created, it would be more opportune to control the sensors manually when this environment is presented. Manual control can be achieved through approaches which may include voice control, gesture control and different forms of physical actuation, the latter of which is present within the preferred embodiment due to its precise control. Otherwise, having them work autonomously to provide information without involvement would be more advantageous. Variations to these approaches may also exist, such as the sensors being triggered automatically once they have perceived the required conditions.

Sensors will typically sense in a periodic fashion as perceived changes may be unlikely to occur constantly, and their rate of sensing may be limited. In some embodiments, sensing may only need to occur once or may be continuous to provide a feed of information in as close to real-time as possible. In situations where snapshots or particular states are sensed, the provision of sensed data in a form of delayed time may be exercised as a number of states may be required to produce gainful data.

The selection of sensors and their configuration will be dependent on their sensing subjects. Sensing a tissue will typically comprise at least one two-dimensional scanner, three-dimensional scanner and hyperspectral or spectral sensors. These should be positioned to surround the tissue with special focus placed on areas of interest, likely relating to the procedure being performed.

Some of these sensors operate in real-time and perform periodic sensing. Other sensors may be excluded from direct operation until a point is reach where personnel prepare the theatre environment for ideal sensing conditions prior to reverting the environment after the sensing has occurred. In both cases it would be opportune for trusted personnel to manually trigger the sensors in addition to their autonomous operation. Manual triggers should comprise physical buttons or a touch screen as to allow for efficient, binary interactions.

Turning again to FIG. 2, the data collection and retrieval step 100 of FIG. 1 is described in greater detail. The surrounding environment and relevant personnel should be prepared 101 for any sensing procedures that may occur, depending on the sensors that are in use. This may involve implicit preparation of the environment to ensure or increase the probability of optimal conditions occurring or temporary explicit modification of the environment if the involved sensors cannot sense efficiently during typical conditions. These modifications may comprise having the personnel move any obstructing equipment and adjust any environmental conditions such as lighting. The preferred embodiment will require implicit preparation and some element of explicit preparation. As orthopaedic surgery is generally time-constrained from both a monetary and medical perspective, reliance on periodic sensors that work around the operation is more plausible than those which require constant changes to the setting, although setup configuration changes occurring a small number of times during a procedure may be acceptable.

The configuration of sensors should be prepared 102 for any sensing procedures that may occur, provided their environment is in such a way that allows this to be possible or at least efficient. This may involve changing the position, alignment and orientation of sensors both independently and in relation to each other. Additional equipment such as stands or platforms may be necessary for these changes. In the preferred embodiment, the sensors will already have the required configuration as part of a pre-constructed system or platform. When the opportunity arises, this system as a whole can be moved into place in a relatively small time-frame, reducing environmental impact and disruption. After preparation 101 and 102 is completed, the sensing procedure 103 can begin.

Sensing will occur based on a set duration which determines the number of repetitions possible based on the particular types and quantities of sensors present in the particular setup arrangement. In embodiments that require the environment and sensor configuration to be adjusted for sensing, these parameters are likely to be constrained by their setting. During orthopaedic surgery, this duration is likely to be only be a few minutes long as time is crucial for success of the surgical procedure, with the number of sensing repetitions being similarly limited as a result. In embodiments that allow for passive sensors, the duration may be dependent on the total lifetime of the subject being sensed or the actions performed in relation to it, allowing for a comparatively higher number of repetitions with respect to the capabilities of the involved sensors. After sensing has occurred, the preparatory measures 101, 102 implemented prior may be reverted if necessary.

Data may also be collected directly through provisions from verified personnel or systems 104 which may include documents, records and databases. In particular embodiments, this would comprise any resources that could provide additional information on the patient or the operation that they are undergoing, such as patient records, medical records and historical operation or surgery data.

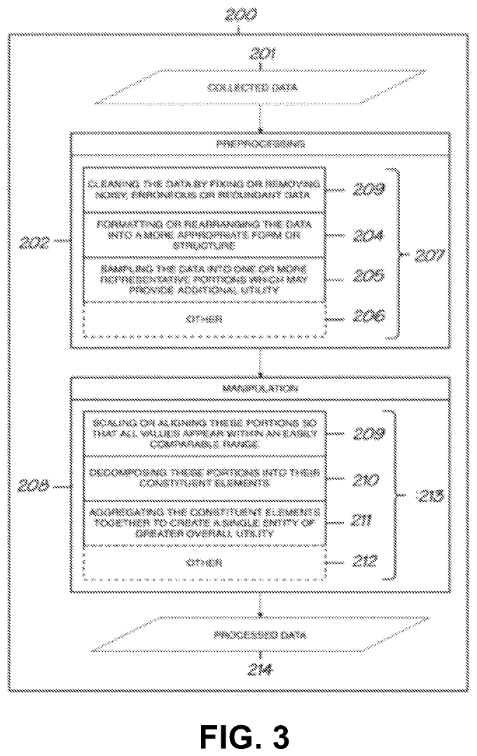

All data sensed and provided will be collected and presented in an easily accessible manner 105 as required by the necessary processing step 200, depicted in detail in FIG. 3. Collection may involve the extraction of data in whichever format is deemed the most usable, generally determined by the sensor it originated from. Sensed data may initially appear in a raw form which must be converted into data in which meaning can be drawn from, typically by an external control unit. Similarly, provided data may appear in a form that cannot be easily accessed, such as paper, which requires input into a digital system to rectify. In particular embodiments, all data would be stored in the same way so that they may be accessed in the same way. This method of storage would ideally be the random-access memory (RAM) of a central system, although a solid-state drive or hard disc may be used instead depending on the raw amount of data and processing speed required for traversal. In other embodiments, a database may be used to store and access this data. It may use strict storage and access guidelines as imposed by SQL or be more flexible and scalable using technology such as NoSQL.

FIG. 3 is a detailed schematic diagram depicting an exemplary data pre-processing and manipulation step 200 of the type depicted in FIG. 1. Data will be processed to transform it into a form of greater utility, generally in terms of both its usability and evaluability.

The data source 201 comprising collected data involved wherein the collected data comprises, at least, the raw sensed data and/or the data provided from trusted personnel or systems, which were either generated from the plurality of sensors located about the subject and/or provided externally. This data should contain enough information to determine the state and class of its associated tissue.

The intent behind processing the data is to best prepare it for training and execution within predictive algorithms and methods including for example machine-learning or artificial neural network systems as would be appreciated by the skilled addressee. This may involve different types of pre-processing and manipulation to transform the data into a form that produces the most benefit with respect to this usage.

Pre-processing the data involves transforming it into a form of superior usability 202 in preparation for and to produce the most utility from subsequent data manipulation. This data may initially be in a somewhat raw form which may contain noise, errors or redundancy. If data containing these flaws is used during normal processing, redundant calculations, inconsistencies or incorrect results may occur. These must therefore be fixed or removed 203 depending on their type and severity.

Noisy data may be defined as data that is partially correct but contains other portions that are corrupt or in error. The proportion between the correct data and that which is in error is an indicator about the type of actions that can be taken in response to it. If only a small amount is in error, then it may be possible to fix this amount based on the correct data, or it could be removed provided that the remaining data provides sufficient benefit in its reduced form. If the amount of incorrect data is large however, then removing the data as a whole is likely the only option.

Erroneous data may be defined as data that is wrong and contains values that cannot possibility exist either through the medium that created it or in relation to surrounding data. Erroneous data cannot be fixed in most scenarios as it typically has no relation to the value that it should have been and is therefore usually removed.

Redundant data may be defined as data which, although not in error, does not add any additional value or benefit to the data set as a whole and only serves to increase its volume and introduce inconsistencies. Redundant data cannot be fixed as it is technically correct and is therefore usually removed or ignored instead.

Removing or fixing data is highly dependent on the origin and format of the data and the severity of the portions in question. Removal is relatively straightforward depending on the format but will leave the remaining data in a reduced state. The data will remain valid in some cases, but others may require additional modifications to achieve this. This may entail combining the remaining data with other sets of reduced data to create complete sets or replacing the data with dummy data that will not affect the end result. The fixing of data in comparison is more difficult and requires knowledge about the expected structure to determine what is missing or wrong so that it can be rectified. Techniques to achieve this are highly dependent on the data itself and may not even be possible. In the preferred embodiment, all redundancy and errors will be directly removed, whist any noise will be fixed if additional benefit can be discerned.

Collected data 201 will typically need to be rearranged and formatted to increase access efficiency and make its storage more logical in terms of processing 204. This is because its initial form will likely be based on the ordering and format of its origin, such as a specific sensor, system or set of personnel, which is suboptimal for manipulation.

In the preferred embodiment, and specifically for the intended predictive approaches, rearrangement will consist of grouping together data which may have established similarities or other relations. This makes accessing or searching for related data or data which cleanly represents a particular aspect or series of aspects easier and more efficient. Formatting will consist of structuring these different groupings in ways that allow different sets of data to be manipulated and analysed simultaneously and subsequently. This will make traversal from one set of data to another related set of data relatively simplistic and computationally inexpensive. Other embodiments will have differing approaches to formatting and arrangement depending on the type of manipulation and subsequent predictive approaches intended for the data.

Collected data 201 may be sampled 205 as part of pre-processing to create different portions which may provide additional utility as opposed to operating based on the data as a whole. Sampling consists of reducing the data pool into one that is more advantageous towards a specific type of usage, such as the data as a whole being reduced to only parts that may be deemed as representative.

In the preferred embodiment, and specifically for the intended predictive approaches, the data will initially be sampled to create a single data pool that is more representative than the data as a whole. This is to say that the utility provided by this representative data pool should be equal to or superior than it was originally. This representative pool will then be split into three different segments. The first and largest segment, known as the training set, will be used for training the predictive algorithms and methods. The second smaller segment, known as the test set, will be used for testing trained predictive approaches. The third smaller segment, known as the validation set, will be used for validating the results of trained predictive approaches that have produced favourable accuracy against the test set.

Other embodiments will likely use a similar sampling approach as is consistent with predictive approaches, although additional customizations may be made depending on their specifics.

Manipulating the data 208 involves transforming it into a form of superior evaluability in preparation for and to produce the most utility from subsequent predictive algorithms or methods. This data may initially be in a form wherein each value exists based on how it was expressed originally. Since each expression will likely be different across the data, achieving an appropriate level of comparability between the different sets may not be viable or may be done so to suboptimal degrees. By scaling or aligning these values to a common point 209, comparability between the different sets increases.

In the preferred embodiment, and specifically for the intended predictive approaches, all values existing within data sets that may be deemed comparable and which have direct or similarly equivalent initial expressions should be scaled. This is because some types of predictive analysis generally work better when all data exists within some known range. It also makes handling the data and distinguishing it easier, especially when presenting the data, should the need arise. Other embodiments will likely use similar scaling techniques which will again be based on their intended predictive algorithms or methods.

Collected data 201 may be reduced, split or decomposed into their constituent elements 210 as part of data manipulation. These individual elements compose the data and can be used to identify which existing features may be more beneficial or representative in comparison to others. This is important for predictive analysis as these types of features generally make good indicators, which may greatly increase their utility.

In the preferred embodiment, and specifically for the intended predictive approaches, data will be decomposed into constituent elements if it can be seen that the individual elements or otherwise features make a considerable contribution in determining the overall description of the data as a whole.

Collected data 201 and the constituent elements derived from it may be aggregated together into a single entity 211 as part of data manipulation. The aggregated entity should provide more utility in comparison to the individual elements or data which were used to create it, although this may not be the case if the decision was made from a storage or computational perspective.

Aggregation approaches are largely dependent on the application, the type and expression of data, and the forms of processing their results will be used with. Simplistic approaches may involve averaging the involved data together whilst more complex ones may involve providing a weighting to each individual element and performing a procedure that processes and combines them based on these weightings. As the amount of information relating to the context of the data and application increases, the complexity and utility granted by these aggregation approaches may do so as well.

In the preferred embodiment, and specifically for the intended predictive approaches, elements should be aggregated together if additional utility will be produced. This means that if an aggregated entity indicates the properties of a set of data better in comparison to the individual elements, then the aggregation should be maintained. It may be performed for all sources of data but will likely be restricted to data of similar origin as different aggregation algorithms may require some amount of similarity to be productive. Other embodiments will likely aggregate data in a similar manner, with their dependencies determining how and to what degree this will occur.

The final processed data 214 will be produced after the collected data 201 has been pre-processed and manipulated. Other pre-processing approaches 206 and manipulation approaches 212 may exist in addition to those mentioned above as would be readily appreciated by the skilled addressee. The ordering and existence of these pre-processing approaches 207 and manipulation approaches 213 may not necessarily reflect the ordering and existence of the approaches herein.

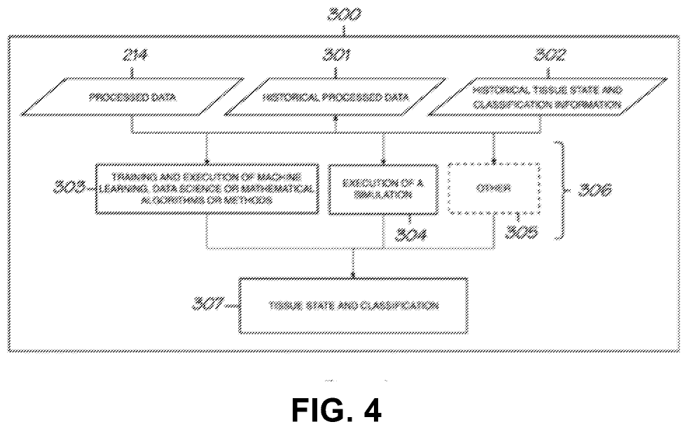

FIG. 4 is a detailed schematic diagram depicting an exemplary data interpretation and information generation step 300 of the type depicted in FIG. 1. This involves using two different data sources within a series of different predictive approaches to generate information and values which may provide insight into the state and classification of a particular tissue

The first data source 214 is the data recently processed (refer to FIG. 3) to produce additional utility during predictive analysis as detailed in FIG. 3. The second data source is the same except that it contains historical processed data 301 that has been generated prior and retrieved 104. These sources will be used as derivable data where indicators and other mapping mechanisms may be found.

The third data source 302, which contains a specific set of values corresponding to each value within the second data source, is the historical data of actual tissue state and classification information. This is used as the ground truth and is what may be predicted.

Predictions may be generated based on the first data source 214 by training and executing different forms of machine learning, data science and mathematical algorithms or methods 303. In the preferred embodiment, this will mainly consist of different supervised approaches. These types of approaches generally operate in two different phases comprising the training phase and the execution phase.

Turning now to FIG. 4 the data interpretation and information generation step 300 of FIG. 1 is described in greater detail. Process 300 includes training of the algorithms for the data interpretation and output in a machine learning or ANN framework. The training phase involves the second and third data sources including the processed data 214 generated by the plurality of sensors located about the subject, and the retrieved historical data 104 wherein each set of data in the second data source 301 maps to a specific set of values in the third 302. It consists of identifying indicators within each set of data that are either partially or majorly responsible for this mapping such that if another set of data contained these same indicators, it would be likely that it would also have the same or similar corresponding values. This will continue until a mapping structure has been developed that will map parsed indicators to values that they most commonly refer to.

The training phase is often performed using different segments of data as opposed to the data as a whole which may include training, testing and validation segments, where the data and corresponding values are known for each. It will initially begin by generating a mapping structure corresponding to only the training segment. The data within the test segment will then be ran through this structure, with the values it returns being compared to the actual known values of the segment. This will provide a measure of accuracy depending on how close the returned values are to the actual ones. If this accuracy is satisfactory (somewhere between 95-100% based on the preferred embodiment) then it is tested again using the validation segment. This is to simulate its performance on real world data as although it has seen the training and test segments previously, the validation segment will remain unknown to it. This ensures that the mapping structure will perform well on all data as opposed to only the test segment, a phenomenon known as over-fitting.

The execution phase involves only the first data source 214 which has no known corresponding values. It initially consists of identifying the same indicators found during the training phase within each set of data in this source. These indicators are then given to the previously created mapping structure to identify the values that they correspond to.

Supervised algorithms or methods differ greatly in their complexity as well as their predictive power and using various types of them concurrently may produce beneficial results in addition to a point of comparison. These algorithms or methods may include linear and polynomial regression, logistic regression, naive bayesian networks, bayesian networks, support vector machines, decision trees, random forests, k-nearest neighbour classifiers and neural networks.

Other embodiments may use different predictive approaches, including unsupervised, semi-supervised and reinforcement approaches. Unsupervised and semi-supervised algorithms or methods are provided a data set and are made to extract meaning from it without any or with little direction as to what it is that they are looking for. This allows unknown information or connections existing within the data to be found which may provide additional utility depending on what they are and their consistency in other data sets.

Reinforcement algorithms or methods may attempt to run a series of calculations with the goal of producing a particular value. They are provided positive or negative stimulus depending on the accuracy of this value in comparison to what it should have been. When provided positive stimulus, they will continue performing the same calculations that they have done and may perform additional ones which are similar to these. When provided negative stimulus they may stop performing their current calculations and try some that are different to varying degrees. A degree of randomness is typically added to these algorithms to give them a starting point, which means that they may require more execution cycles to reach a satisfactory result in comparison to the prior predictive analysis approaches.

Predictions may be generated based on processed data 214 by running a simulation 304 that involves the different types of scenarios and conditions which may affect the state of the tissue. These types of instances will likely be simulated mathematically with probabilistic measures added to account for situations that are not currently determinative.

In the preferred embodiment, the simulation will be designed around the different procedures or techniques which may occur intraoperatively and their associated effects on the tissue. It will be provided two main sources of data.

The first source will be processed data 214 that contains various information relating to the state and class of the tissue. This will be used as a foundation platform for the simulation to begin its processing.

The second source 301 will be information similar to the first but having occurred after a particular event or procedure where any associated variables are known. This information will indicate the effect that these variables may have participated in creating.

Currently the simulation has been referred to as singular, but this may not be the case if additional benefit can be found by having multiple different simulations or by dividing a single simulation into multiple individual simulations that each have their own purpose or predictive goal. Considering the complexity that is usually involved, division may be advantageous at least from a development and production point of view.

Other embodiments may utilise different simulations depending on their context and application. This would likely be dependent on their subject and the desired results of the simulation.

Predictions generated will be used to provide insight into information relating to the state and classification of tissue 307. These two sets of identifying information comprise various properties and characteristics which generate their value as a whole. They can be used individually or together to determine and inform the results of various procedures or events surrounding specific tissue.

Tissue state comprises a series of descriptors that provide information relating to tissue health. These descriptors generally revolve around a particular aspect of the tissue and may change over time or after significant events. Composition is one such descriptor that describes the types of minerals that may comprise the tissue. The different minerals and their abundances generally make a reliable indicator as to the health and age of a specific tissue and notably differ when variations to these properties are present. This is especially useful when gauging tissue density, which is defined by how tightly packed these minerals, or at least specific minerals, are in relation to each other. Heat consistency is another such descriptor which describes the temperature of the tissue and how this is distributed across it. Measuring heat consistency is often a good way to monitor how the tissue is being affected when performing operations such as osteotomies. Other state descriptors may include hydration which may describe the water content existing within the tissue that can be useful in measuring the impact of any prior osteotomies; necrosis which describes the death of tissue cells which may have been caused by the method of osteotomy or internal issues within the body; colouration which describes the particular colour that the tissue exhibits wherein any variations typically cannot be discerned without advanced visual sensors; and reflectance which describes how much and what colours the tissue can actively reflect. Tissue state information or descriptors may be used individually, within a selective set or as a whole to determine or predict the results that a particular procedure or set of variables has on the tissue.

Tissue classification comprises defining the type of a particular tissue and any specializations that can be derived on top of that. This may comprise bone, cartilage, fat, ligament, muscle and meniscus. It may be used to define the type of tissue either individually or as part of a selected area or region and can reinforce or further inform conclusions made in conjunction with state information.

Other predictive approaches and resultant information may exist external to those explicitly outlined herein 305. Predictive approaches may not necessarily only be executed singularly, they may also be executed concurrently and sequentially if reason exists to do so 306.

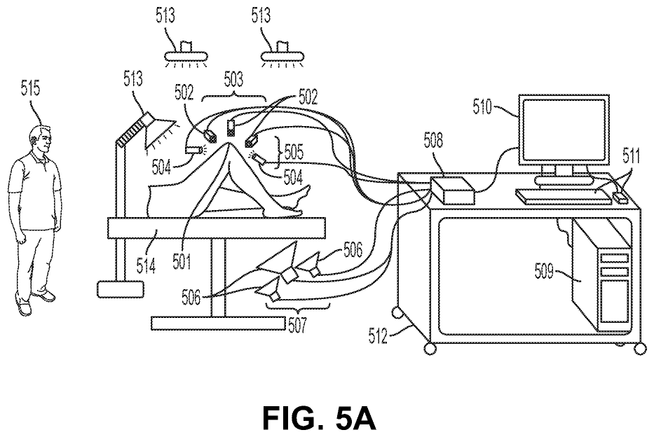

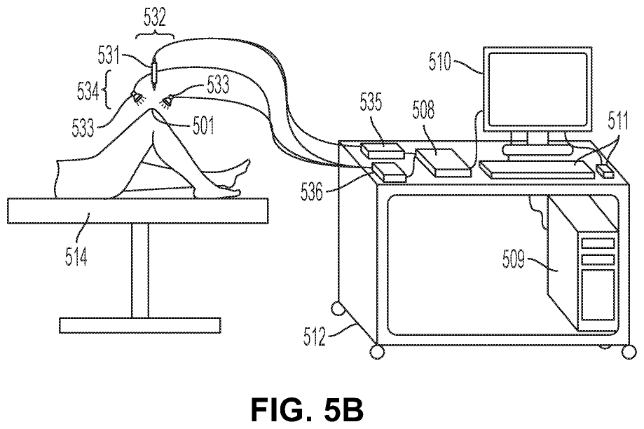

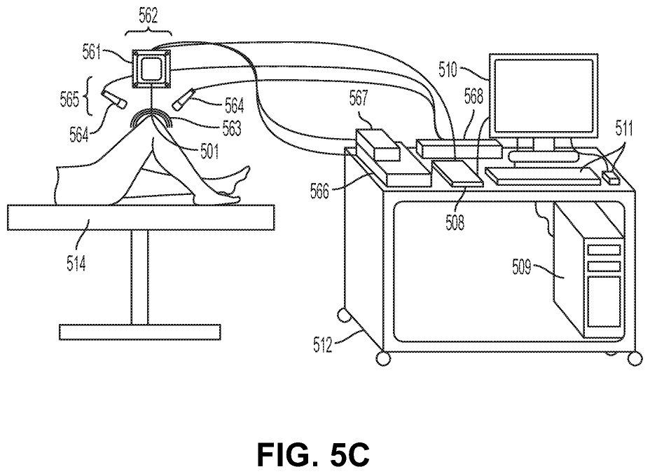

FIG. 5A illustrates generally a basic embodiment for tissue state and class analysis and the intraoperative environment in which it is used. It includes a plurality of sensors 502 arranged about the subject joint 501 of the patient (in the illustrated case, the patient's right knee). The plurality of sensors 502 may comprise different types and models and may not necessarily have uniformity. The number and arrangement 503 of these sensors may differ depending on the subject 501 and the particularities of the application in analysing a particular subject.

Some sensors may not work independently and may instead require additional sensors or assistant devices 504 such as, for example, light sources. These devices may comprise different types and models and may also differ in their number and arrangement 505 in accordance with requirements.

Sensors 502 may not generate data directly and may instead rely on a separate capture device 506 to handle or support this generation process to varying degrees. These capture devices 506 may comprise different types and models and may also differ in their number and arrangement 507 accordance with requirements, with these specifics likely being dependent on their associated sensors 502.

In particular embodiments, the subject of these sensors and any additional equipment will be articular tissue derived from a knee 501 or different joints (for example, hip, elbow, shoulder etc.) of the patient upon which an orthopaedic procedure is being performed. This may however be any other form of tissue existing within any other form of surgery. The illustrated arrangement provides an example as to how the sensors and associated equipment may be positioned relative to the subject 501 in the present embodiment. By positioning the sensors around the subject 501 through many different angles, it can be assumed that a high level of visibility may be achieved.

Temporary or permanent adjustments or modifications to the intraoperative environment during associated medical procedures or surgeries may be necessary to achieve plausible sensing conditions or increased sensing accuracy. This may comprise adjustments to immediate environmental conditions such as lighting 513, changes to the position or arrangement of the subject (e.g. by movement of the surgical bed/table 514 relative to the sensors 502), and the removal or repositioning of personnel 515 within the environment.

Similar adjustments or modifications may need to be made for the sensors 502 and their associated equipment such as for example, computers 509 and monitors 510, controllers 508 etc. Depending on the type of medical procedure or surgery, this associated equipment may need to be outside of the operating environment completely and reintroduced at specific intervals when it is deemed necessary or safe for the patient. In particular embodiments, the associated equipment will be positioned safely around the subject as to not disturb or hamper the vision of related surgical or support personnel. Once a point in the surgery has been reached or their involvement is deemed necessary, the associated equipment may be moved closer or to a more opportune location prior to being moved back. Other embodiments may not require any repositioning, with the sensors existing simply as passive devices or they may take precedence such that the personnel will be moved into position at points determined by the system with the sensors given priority otherwise.

All data and information provided by the sensors 502 is advantageously received by a peripheral controller 508. This controller 508 may perform any amount of processing required to compile the data into a form that may be delivered to and interpreted by the remainder of the system 509.

The collected data will be processed within the system 509 responsible for executing the analysis process. As defined through the processes depicted in FIGS. 1-3, this processing will comprise pre-processing and manipulating the data, generating a means of predictive analysis based on this data and executing this analysis to derive meaningful results.

All generated information will be displayed through a form of communicative medium that intended personnel can interpret. In the preferred embodiment, this will be an LCD screen, such as that provided by a computer monitor 510. In other embodiments, this may comprise sound recordings, representative lighting or printed material.

Interaction may be possible with the selected communicative medium based on actions provided by the user. In the preferred embodiment, this will comprise a keyboard and mouse 511 which will enable the LCD screen 510 to be interacted with in a natural and familiar way. It may also allow the forms of possible interaction to be more complex with a larger range of options. In other embodiments, this may comprise voice commands, gesture commands and a touch screen interface.

System components may be arranged together within a singular system or container 512 such that all interactions with the individual components will comprise an interaction with this container. In the preferred embodiment, a structure housing all of these components will be developed such that they may be moved, adjusted and associated with each other in a clear and simplistic manner. In other embodiments, they may be individually positioned and arranged such that each exists independently and can be adjusted without necessarily effecting the state of the remaining components.

FIG. 5B illustrates generally an implementation of the basic embodiment of FIG. 5A which utilises spectral or hyperspectral sensors for intraoperative tissue state and class analysis. It includes an array of spectral or hyperspectral sensors 531 which may comprise different types and models and not necessarily have uniformity. They may have any singular or combination of bands available within the electromagnetic spectrum, with this likely being determined by the bands that evoke the greatest response from their subject. The number and arrangement of these spectral sensors may differ 532 depending on their subject and application. In the preferred embodiment, each subject will have a single corresponding spectral sensor unless benefit exists in having multiple, such as in scenarios where multiple bands are required but cannot be contained within a single sensor.

Spectral or hyperspectral sensors 531 generally require a specific type or level of light to produce useful information. Although this light may be achieved from natural lighting or from those introduced by the surgical environment, specialised sources that can be controlled or which exhibit a specific form of light may be necessary. These light sources 533 typically consist of a generator 536 such as an LED, a transmission medium which is usually a type of fibre, and a light output diffuser. They may comprise UV, visible and infrared lights of variable brightness and strength and may differ in type, model, number and arrangement 534. They may exist independently or be coupled within other hardware such as a spectral or hyperspectral sensor. In the preferred embodiment, each spectral or hyperspectral sensor will have as many light sources as necessary to illuminate their subject and increase their accuracy.

Temporary adjustments to the environment may be necessary to allow the spectral or hyperspectral sensors 531 to perform as intended. Since they operate based on light, this will typically comprise adjustments to the lighting which may involve dimming external lights to increase the effectiveness of specialised lights, removing any obstacles in their vision or of which may cause shadows and decreasing the distance between them and the subject to encourage light penetration.

Light sensed by spectral or hyperspectral sensors 531 may need to be passed through a spectrometer 535 before it can be utilised. This is a device that takes light and measures the intensity as an array of separate colours within the possible available bands. Each spectrometer will be matched with at least one spectral or hyperspectral sensor with the nature of this correspondence being dependent on the type and model of all involved components.

The light sources 533 and spectrometers 535 will be controlled by and communicate with single or multiple peripheral controllers 508 depending on the application and sensor arrangement. This controller will likely perform some form of preliminary processing to transform the received data into a more usable form prior to passing it to the remainder of the system 509.

The preferred embodiment will likely possess some form of spectral or hyperspectral sensing in order to distinguish between the different types of tissue as each one may reflect and absorb differing amounts and frequencies of light. These amounts may also be influenced by variables relating to the state such as hydration and necrosis which may also be discerned through these sensors.

FIG. 5C illustrates generally an implementation of the basic embodiment of FIG. 5A which utilises acoustic sensors for intraoperative tissue state and class analysis. It includes an array of acoustic sensors 564 which may comprise different types and models and not necessarily have uniformity. They may have any possible sampling rate, with this likely being determined by the sound evoked from physically impacting their subject. The number and arrangement of these acoustic sensors may differ 565 depending on their subject and application. In the preferred embodiment, each subject will be surrounded by multiple acoustic sensors to sample the sound produced from many different angles.

Acoustic sensors can only perceive sound from a static tissue if it is impacted by something 563. The actuator 561 of this impact must therefore produce it in a constant and measured fashion as if the amount of force or any other variables differ, a different sound will be produced. Although the signature of this sound will likely be the same, having an actuator that will reduce any inconsistencies will be advantageous. The number and arrangement of these actuators may differ 562 depending on the application as creating sound from multiple different angles provided that the actuators are appropriately synchronised may be beneficial. In the preferred embodiment, as illustrated, the actuator will comprise a single laser system that will emit a measured and consistent single pulse of light into the tissue to produce sound. By using a highly controlled actuator, the resulting sound frequencies will be more consistent. This is important when dealing with subjects whose potentially differing states may produce subtly similar sounds such that even relatively small amounts of actuator variance may affect the final result.

Temporary adjustments to the environment may be necessary to allow the acoustic sensors to perform as intended. Since they operate based on sound, moving the sensors closer to the source of this sound will typically give that specific audio frequency more prevalence in comparison to other sounds produced in the environment. However, this also means that data returned from these sensors will likely vary based on their arrangement, although will still maintain the same or a similar signature. This makes it likely that large movements of the sensors would provide little benefit in comparison to placing them statically. In the preferred embodiment, the sensors will be placed statically at the beginning of a medical procedure or surgery and remain there until they are no longer needed. Their development will therefore likely focus on identifying signatures instead of raw acoustic signals.

The actuator 561 or actuators used will require single or multiple devices to control their output and triggering. The illustrated laser system will require two different devices, with the configuration and selection of them being dependent on the strength and type of laser. The first of these is the laser current source 566 which generates the required current internally prior to sending it to the laser. The second is the laser chiller 567 which will cool the laser between pulses to ensure that it does not overheat and damage itself or its surroundings. The preferred embodiment will require both of these components.

Acoustic signals monitored by the sensors will need to be received and transformed into a form that can be interpreted which is typically handled by an audio interface or receiver 568. The configuration of the different sensors including their rate and sensitivity can be controlled through this system. The preferred embodiment will use a single receiver with all involved acoustic sensors being attached to it such that their signals may be interpreted as a whole instead of individually as they will likely perceive the same subject but from different angles and positions.

All components may be attached to single or multiple peripheral controllers 508 depending on the application and sensor arrangement. This controller will likely perform some form of preliminary processing to transform the received data into a more usable form prior to passing it to the remainder of the system 509.

The preferred embodiment will likely possess some form of acoustic sensing in order to determine specific types of tissue state information such as density and hydration. The type of tissue can then be derived from this or used as reinforcement for conclusions developed through other sensing methods.

The features presented herein may be performed electronically through any capable system or machine that can complete them within any restrictions applied by their particular application. This may be performed online, offline or in a capacity that relies on some combination of the two.

Data extracted or generated as a result of the features presented herein may be stored electronically which can be done offline, online, or through some combination of the two. This may be accessed immediately or in a delayed time frame for retrieval, processing and any other form of usage. All types of data may be stored and may be maintained singularly, intermittently, routinely or through any other timing paradigm.

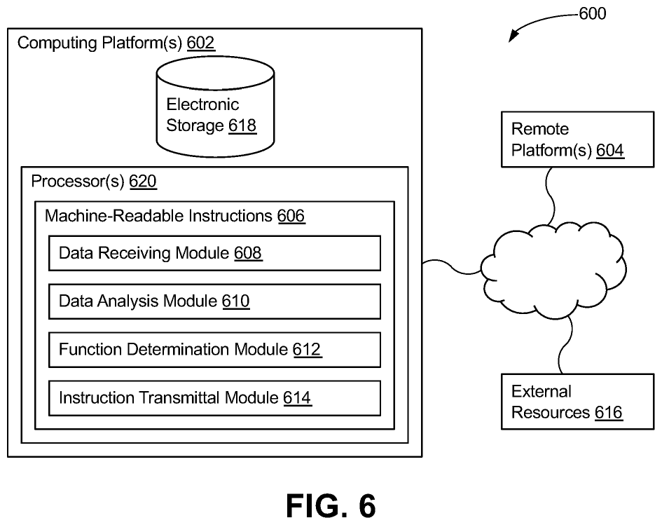

FIG. 6 illustrates a system 600 configured for robot-assisted laser osteotomy, in accordance with one or more implementations. In some implementations, system 600 may include one or more computing platforms 602. Computing platform(s) 602 may be configured to communicate with one or more remote platforms 604 according to a client/server architecture, a peer-to-peer architecture, and/or other architectures. Remote platform(s) 604 may be configured to communicate with other remote platforms via computing platform(s) 602 and/or according to a client/server architecture, a peer-to-peer architecture, and/or other architectures. Users may access system 600 via computing platform(s) 602 and/or remote platform(s) 604.

Computing platform(s) 602 may be configured by machine-readable instructions 606. Machine-readable instructions 606 may include one or more instruction modules. The instruction modules may include computer program modules. The instruction modules may include one or more of data receiving module 608, data analysis module 610, function determination module 612, instruction transmittal module 614, and/or other instruction modules.

Data receiving module 608 may be configured to receive osteotomy data obtained in connection with robot-assisted laser osteotomy. The osteotomy data may be associated with a specific robot-assisted laser osteotomy operation performed on a specific subject. The osteotomy data may be aggregated from among a plurality of robot-assisted laser osteotomy operations performed on a plurality of subjects. By way of non-limiting example, the osteotomy data may include one or more of device data, medical data, and/or outcome data.

By way of non-limiting example, the device data may include one or more of video data, laser data, operating data, hyperspectral data, joint kinematics data and/or orthosensor data. The laser data may include one or more of laser pulse energy, laser frequency, laser fluence, water delivery and/or ablation patterns. Joint kinematics data may be obtained via a force and/or strain sensor. Soft tissue damage may be detected and/or measured based on the hyperspectral data. Bone quality may be determined based on the hyperspectral data. The orthosensor data may be obtained via an orthosensor which, in functionality, may be capable of sensing soft tissue loading and/or balancing. The orthosensor may include a poly insert and/or other material. By way of non-limiting example, the medical data may include one or more of narrative textual data, pre-operative medical scans, numerical measurements, recorded signals, images, pain scores, and/or demographics. By way of non-limiting example, the outcome data may include one or more of subject outcome, survivorship, post-operative medical scans, bone surface data before osteotomy, soft-tissue loading and/or balancing, and/or bone surface data after osteotomy.

By further way of non-limiting example, the device data may include certain categories of data including preoperative data, intra-operative data, post-operative (inpatient) data, and post-operative (outpatient) data. Preoperative data may include patient clinical history (for example, diagnosis, pain, side (left/right/bilateral), aetiology, comorbidities, and risk factors). Intra-operative data may include surgical approach, time of initial incision, physical landmark registration (by surgeon), digital landmark registration (by robot-assisted laser osteotomy system), surface scan (from robot-assisted laser osteotomy system), surgeon-selected parameters (including, but not limited to, selected post-cut alignment, and implant type and sizing), each ablation plan and scan (of the robot-assisted laser osteotomy system), implant fit analysis, soft tissue balancing, digital sensor data (for example, but not limited to, pressure, load etc.), robot-assisted laser osteotomy system settings (for example, but not limited to, laser parameter, water used etc.), implant insertion time, skin closure time, anaesthetic details (for example, but not limited to, drugs used, pain medication used, infection control, time under etc.). Post-operative (inpatient) data may include postoperative imaging (for example, but not limited to CT, EOS scan, X-ray etc.), length of hospital stay, pain levels and medication, and complications (if any). Post-operative (outpatient) data may include joint registry survivorship data, and patient-reported outcome measures (if any).

Data analysis module 610 may be configured to analyse the osteotomy data to determine one or more attributes associated with osteotomy. The one or more attributes may be associated with a specific robot-assisted laser osteotomy operation performed on a specific subject. The one or more attributes may be aggregated from among a plurality of robot-assisted laser osteotomy operations performed on a plurality of subjects. By way of non-limiting example, the one or more attributes may include one or more of a bone quality, a cut quality, a digital twin, a tissue analysis, a hyperspectral result, and/or a laser pulse ablation measurement. By way of non-limiting example, the bone quality may relate to the mechanical and/or biological properties of the bone. This may include, but is not limited to, bone density, bone strength, cell health and/or bone composition. By way of non-limiting example, the cut quality may relate to one or more of surface roughness, cut precision, kerf width, striation patterns, and/or cutting geometry. By way of non-limiting example, the digital twin may include one or more of a 3D representation of the joint, tissue differentiation of the biological structures, location of the cut surfaces and/or location of the robotic system. The digital twin may be provided as a render, may be used as an input for machine learning and/or may be provided to a surgeon during osteotomy. The tissue analysis may include a hyperspectral analysis of tissue. The tissue analysis may be provided as a render. The tissue analysis may be used as an input for machine learning. The tissue analysis may be provided to a surgeon during osteotomy.

Differences in laser pulse ablation between subjects may be determined based on the one or more attributes. The differences in laser pulse ablation between subjects may include the differences between laser pulses required to ablate a given amount of tissue. The differences in laser pulse ablation between subjects may be a basis for determining one or both of bone quality and/or time to completion. By way of non-limiting example, the differences in laser pulse ablation between subjects may be a basis for determining a safe set of parameters that is safe, fast, and clean for. The differences in laser pulse ablation between subjects may be a basis for determining a customized laser dose based on a given subjects own body and conditions.

Data-informed implant designs may be designed based on one or more attributes. The data-informed implant designs may be tailored for individual subjects and may include a set of standard implant sizes. Data-informed implant selection may facilitate selecting a specific implant from a set of standard implants for a specific subject.

Function determination module 612 may be configured to determine, based on the one or more attributes, one or more functions associated with osteotomy. The one or more functions may include one or both of an intraoperative function or a post-operative function. The one or more functions may facilitate surgical optimisations achieved with the benefit of prior data of procedures (e.g., with a broad range of data across patients of different ages, sexes, ethnic background, and pre-existing conditions). In some implementations, the one or more functions may utilize pre-operative and interoperative data as a basis to optimise joint alignment, implant selection, and/or rehabilitation programs.

By way of non-limiting example, the intraoperative function may include one or more of a fully autonomous surgery function, an implant positioning guidance function, a point of no return warnings function, and/or a time to completion estimates function. The fully autonomous surgery function may facilitate a trained machine learning system in autonomously performing an osteotomy procedure. The fully autonomous surgery function may affect laser beam position, geometry, and/or power. The fully autonomous surgery function may affect an ablation rate, a geometry of ablated volume, and/or other ablation characteristics. The implant positioning guidance function may facilitate determining an optimal position of an implant. Determining an optimal position of an implant may include dynamically aligning a joint using a digital twin of the joint under different scenarios. The soft tissue damage detection function may facilitate notification of a surgeon to potential damage to soft tissue structures throughout the osteotomy process. The digital twin of the joint may be determined based on a functional magnetic resonance imaging scan. The point of no return warnings may function facilitates provisioning of a warning to a surgeon as a point of no return in bone ablation is approaching and/or has passed. The time to completion may estimate function facilitates providing a surgeon with a time estimate for how much longer a tissue resection process will take.

By way of non-limiting example, the post-operative function may include one or more of an operation outcome reports function, a data-informed rehabilitation plans function, data-informed implant designs, data-informed implant selection, a certification via verifiable use statistics function, and/or a collated results function. The operation outcome report function may facilitate generation of an operation outcome report from intra-operatively collected data. By way of non-limiting example, the operation outcome report may include one or more of data associated with bone tissue before osteotomy and bone tissue after osteotomy, implant-bone fit, cut quality, soft tissue damage, joint balancing, spectral analysis of bone, and/or bone quality. The operation outcome report may be used as an input for fully autonomous surgery and/or as a training input for fully autonomous surgery. The data-informed rehabilitation may plan function facilitates providing recommendations regarding rehabilitation of a given subject based on collected intraoperative information. Data-informed implant designs may be designed based on the one or more attributes. The data-informed implant designs may be tailored for individual subjects. The data-informed implant designs may include a set of standard implant sizes. Data-informed implant selection may facilitate selecting a specific implant from a set of standard implants for a specific subject. The surgeon certification function may facilitate issuance of a certification provided to a surgeon after completing a certain number of procedures. The certification via verifiable use statistics may function facilitates issuance of a certification provided to surgeons after completing a certain number of procedures. The collated results function may facilitate collating osteotomy data.

Instruction transmittal module 614 may be configured to transmit, via a network, to a remote computing platform 604, one or more instructions to perform the one or more functions. The one or more functions may be interpreted and carried out by a hardware processor on the remote computing platform 604.

In some implementations, computing platform(s) 602, remote platform(s) 604, and/or external resources 616 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which computing platform(s) 602, remote platform(s) 604, and/or external resources 616 may be operatively linked via some other communication media.

A given remote platform 604 may include one or more processors configured to execute computer program modules. The computer program modules may be configured to enable an expert or user associated with the given remote platform 604 to interface with system 600 and/or external resources 616, and/or provide other functionality attributed herein to remote platform(s) 604. By way of non-limiting example, a given remote platform 604 and/or a given computing platform 602 may include one or more of a server, a desktop computer, a laptop computer, a handheld computer, a tablet computing platform, a NetBook, a Smartphone, a surgical instrument, and/or other computing platforms.

External resources 616 may include sources of information outside of system 600, external entities participating with system 600, and/or other resources. In some implementations, some or all of the functionality attributed herein to external resources 616 may be provided by resources included in system 600.

Computing platform(s) 602 may include electronic storage 618, one or more processors 620, and/or other components. Computing platform(s) 602 may include communication lines, or ports to enable the exchange of information with a network and/or other computing platforms. Illustration of computing platform(s) 602 in FIG. 6 is not intended to be limiting. Computing platform(s) 602 may include a plurality of hardware, software, and/or firmware components operating together to provide the functionality attributed herein to computing platform(s) 602. For example, computing platform(s) 602 may be implemented by a cloud of computing platforms operating together as computing platform(s) 602.

Electronic storage 618 may comprise non-transitory storage media that electronically stores information. The electronic storage media of electronic storage 618 may include one or both of system storage that is provided integrally (i.e., substantially non-removable) with computing platform(s) 602 and/or removable storage that is removably connectable to computing platform(s) 602 via, for example, a port (e.g., a USB port, a firewire port, etc.) or a drive (e.g., a disc drive, etc.). Electronic storage 618 may include one or more of optically readable storage media (e.g., optical discs, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 618 may include one or more virtual storage resources (e.g., cloud storage, a virtual private network, and/or other virtual storage resources). Electronic storage 618 may store software algorithms, information determined by processor(s) 620, information received from computing platform(s) 602, information received from remote platform(s) 604, and/or other information that enables computing platform(s) 602 to function as described herein.

Processor(s) 620 may be configured to provide information processing capabilities in computing platform(s) 602. As such, processor(s) 620 may include one or more of a digital processor, an analogue processor, a digital circuit designed to process information, an analogue circuit designed to process information, a state machine, and/or other mechanisms for electronically processing information. Although processor(s) 620 is shown in FIG. 6 as a single entity, this is for illustrative purposes only. In some implementations, processor(s) 620 may include a plurality of processing units. These processing units may be physically located within the same device, or processor(s) 620 may represent processing functionality of a plurality of devices operating in coordination. Processor(s) 620 may be configured to execute modules 608, 610, 612, and/or 614, and/or other modules. Processor(s) 620 may be configured to execute modules 608, 610, 612, and/or 614, and/or other modules by software; hardware; firmware; some combination of software, hardware, and/or firmware; and/or other mechanisms for configuring processing capabilities on processor(s) 620. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 608, 610, 612, and/or 614 are illustrated in FIG. 6 as being implemented within a single processing unit, in implementations in which processor(s) 620 includes multiple processing units, one or more of modules 608, 610, 612, and/or 614 may be implemented remotely from the other modules. The description of the functionality provided by the different modules 608, 610, 612, and/or 614 described below is for illustrative purposes, and is not intended to be limiting, as any of modules 608, 610, 612, and/or 614 may provide more or less functionality than is described. For example, one or more of modules 608, 610, 612, and/or 614 may be eliminated, and some or all of its functionality may be provided by other ones of modules 608, 610, 612, and/or 614. As another example, processor(s) 620 may be configured to execute one or more additional modules that may perform some or all of the functionality attributed below to one of modules 608, 610, 612, and/or 614.

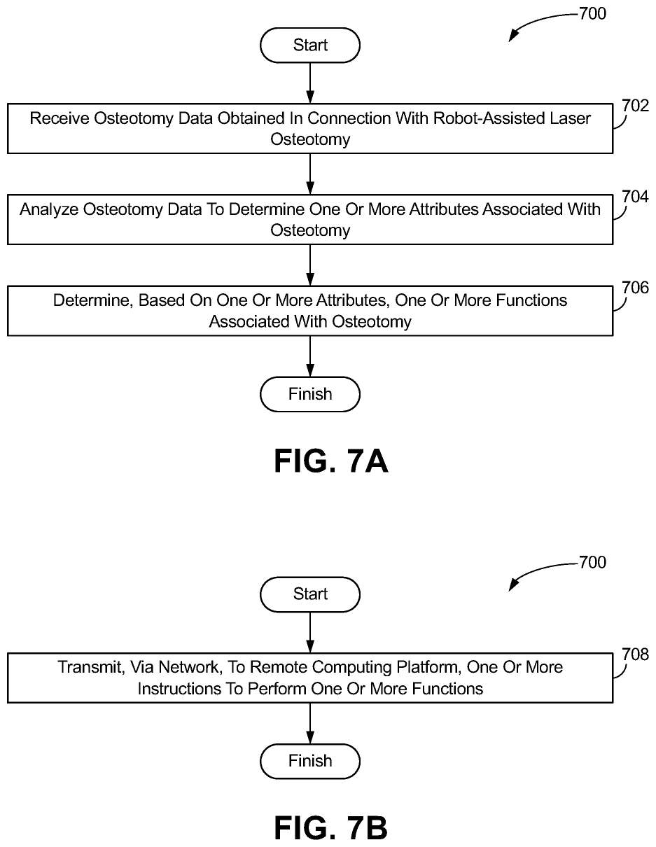

FIGS. 7A and/or 7B illustrates a method 700 for robot-assisted laser osteotomy, in accordance with one or more implementations. The operations of method 700 presented below are intended to be illustrative. In some implementations, method 700 may be accomplished with one or more additional operations not described, and/or without one or more of the operations discussed. Additionally, the order in which the operations of method 700 are illustrated in FIGS. 7A and/or 7B and described below is not intended to be limiting.

In some implementations, method 700 may be implemented in one or more processing devices (e.g., a digital processor, an analogue processor, a digital circuit designed to process information, an analogue circuit designed to process information, a state machine, and/or other mechanisms for electronically processing information). The one or more processing devices may include one or more devices executing some or all of the operations of method 700 in response to instructions stored electronically on an electronic storage medium. The one or more processing devices may include one or more devices configured through hardware, firmware, and/or software to be specifically designed for execution of one or more of the operations of method 700.

FIG. 7A illustrates method 700, in accordance with one or more implementations.

A step 702 may include receiving osteotomy data obtained in connection with robot-assisted laser osteotomy. The osteotomy data may include one or more of device data, medical data, and/or outcome data. Step 702 may be performed by one or more hardware processors configured by machine-readable instructions including a module that is the same as or similar to data receiving module 608, in accordance with one or more implementations.

A step 704 may include analysing the osteotomy data to determine one or more attributes associated with osteotomy. Step 704 may be performed by one or more hardware processors configured by machine-readable instructions including a module that is the same as or similar to data analysis module 610, in accordance with one or more implementations.

A step 706 may include determining, based on the one or more attributes, one or more functions associated with osteotomy. The one or more functions may include one or both of an intraoperative function and/or a post-operative function. Step 706 may be performed by one or more hardware processors configured by machine-readable instructions including a module that is the same as or similar to function determination module 612, in accordance with one or more implementations.

FIG. 7B illustrates method 700, in accordance with one or more implementations.

A step 708 may include further including transmitting, via a network, to a remote computing platform, one or more instructions to perform the one or more functions. The one or more functions may be interpreted and carried out by a hardware processor on the remote computing platform. Step 708 may be performed by one or more hardware processors configured by machine-readable instructions including a module that is the same as or similar to instruction transmittal module 614, in accordance with one or more implementations.

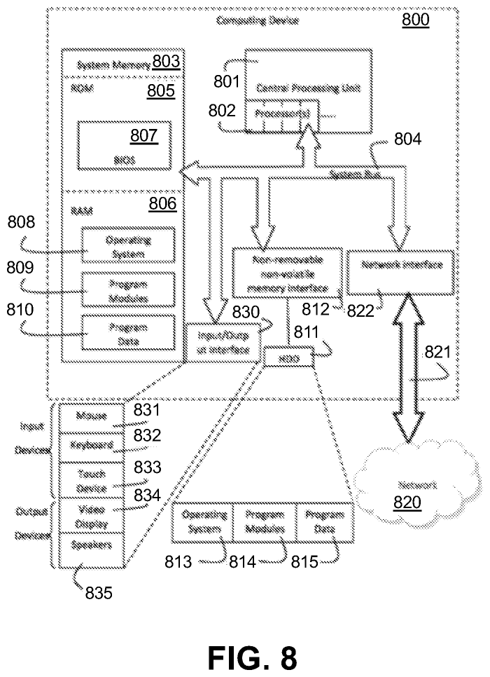

Embodiments disclosed herein may be implemented using a computing device/computer system 800, such as that shown in FIG. 8 wherein the processes may be implemented as software, such as one or more application programs executable within the computing device 800. In particular, the steps of methods depicted in FIGS. 1-4 and 7 are effected by instructions in the software that are carried out within the computer system 800. The instructions may be formed as one or more code modules, each for performing one or more particular tasks. The software may also be divided into two separate parts, in which a first part and the corresponding code modules performs the described methods and a second part and the corresponding code modules manage a user interface between the first part and the user. The software may be stored in a computer readable medium, including the storage devices described below, for example. The software is loaded into the computer system 800 from the computer readable medium, and then executed by the computer system 800. A computer readable medium having such software or computer program recorded on it is a computer program product. The use of the computer program product in the computer system 800 preferably effects an advantageous apparatus for robot-assisted laser osteotomy including post-operative care and evaluation of orthopaedic surgical procedures.

With reference to FIG. 8, an exemplary computing device 800 is illustrated. The exemplary computing device 800 can include, but is not limited to, one or more central processing units (CPUs) 801 comprising one or more processors 802, a system memory 803, and a system bus 804 that couples various system components including the system memory 803 to the processing unit 801. The system bus 804 may be any of several types of bus structures including a memory bus or memory controller, a peripheral bus, and a local bus using any of a variety of bus architectures.

The computing device 800 also typically includes computer readable media, which can include any available media that can be accessed by computing device 800 and includes both volatile and non-volatile media and removable and non-removable media. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile discs (DVD) or other optical disc storage, magnetic cassettes, magnetic tape, magnetic disc storage or other magnetic storage devices, or any other medium which can be used to store the desired information and which can be accessed by the computing device 800. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 803 includes computer storage media in the form of volatile and/or non-volatile memory such as read only memory (ROM) 805 and random-access memory (RAM) 806. A basic input/output system 807 (BIOS), containing the basic routines that help to transfer information between elements within computing device 800, such as during start-up, is typically stored in ROM 805. RAM 806 typically contains data and/or program modules that are immediately accessible to and/or presently being operated on by processing unit 801. By way of example, and not limitation, FIG. 8 illustrates an operating system 808, other program modules 809, and program data 810.

The computing device 800 may also include other removable/non-removable, volatile/non-volatile computer storage media. By way of example only, FIG. 8 illustrates a hard disc drive 811 that reads from or writes to non-removable, non-volatile magnetic media. Other removable/non-removable, volatile/non-volatile computer storage media that can be used with the exemplary computing device include, but are not limited to, magnetic tape cassettes, flash memory cards, digital versatile discs, digital video tape, solid state RAM, solid state ROM, and the like. The hard disc drive 811 is typically connected to the system bus 804 through a non-removable memory interface such as interface 812.

The drives and their associated computer storage media discussed above and illustrated in FIG. 8, provide storage of computer readable instructions, data structures, program modules and other data for the computing device 800. In FIG. 8, for example, hard disc drive 811 is illustrated as storing an operating system 813, other program modules 814, and program data 815. Note that these components can either be the same as or different from operating system 808, other program modules 809 and program data 810. Operating system 813, other program modules 814 and program data 815 are given different numbers hereto illustrate that, at a minimum, they are different copies.

The computing device also includes one or more input/output (I/O) interfaces 830 connected to the system bus 804 including an audio-video interface that couples to output devices including one or more of a video display 834 and loudspeakers 835. Input/output interface(s) 830 also couple(s) to one or more input devices including, for example a mouse 831, keyboard 832 or touch sensitive device 833 such as for example a smartphone or tablet device.

In relevance to the descriptions below, the computing device Z00 may operate in a networked environment using logical connections to one or more remote computers. For simplicity of illustration, the computing device Z00 is shown in FIG. 8 to be connected to a network 820 that is not limited to any particular network or networking protocols, but which may include, for example Ethernet, Bluetooth or IEEE 802.X wireless protocols. The logical connection depicted in FIG. 8 is a general network connection 821 that can be a local area network (LAN), a wide area network (WAN) or other network, for example, the internet. The computing device 800 is connected to the general network connection 821 through a network interface or adapter 822 which is, in turn, connected to the system bus 804. In a networked environment, program modules depicted relative to the computing device 800, or portions or peripherals thereof, may be stored in the memory of one or more other computing devices that are communicatively coupled to the computing device 800 through the general network connection 821. It will be appreciated that the network connections shown are exemplary and other means of establishing a communications link between computing devices may be used.

It should be understood that the features presented herein and the different processes that they contain do not necessarily need to be performed in the described order nor do they require a specific environment or situation. Their ordering, nature, preparation and execution may be dependent on numerous circumstances as is typically the case with medically applicable inventions or methods.

It will be appreciated by those skilled in the art that variations and modifications to the invention described herein will be apparent without departing from the spirit and scope thereof. The variations and modifications as would be apparent to persons skilled in the art are deemed to fall within the broad scope and ambit of the invention as herein set forth.

Future patent applications may be filed in Australia or overseas on the basis of, or claiming priority from, the present application. It is to be understood that the following provisional claims are provided by way of example only and are not intended to limit the scope of what may be claimed in any such future application. Features may be added to or omitted from the provisional claims at a later date so as to further define or redefine the invention or inventions.

1. A system configured for robot-assisted laser osteotomy, the system comprising: one or more hardware processors configured by machine-readable instructions to:
receive osteotomy data obtained in connection with robot-assisted laser osteotomy, the osteotomy data including one or more of device data, medical data, and/or outcome data;
analyse the osteotomy data to determine one or more attributes associated with osteotomy; and
determine, based on the one or more attributes, one or more functions associated with osteotomy, the one or more functions including one or both of an intraoperative function or a post-operative function.

2. (canceled)

3. The system of claim 1, wherein the device data includes one or more of video data, laser data, operating data, hyperspectral data, and/or orthosensor data.

4. The system of claim 3, wherein soft tissue damage is detected and/or measured based on the hyperspectral data.

5. The system of claim 3, wherein bone quality is determined based on the hyperspectral data.

6. The system of claim 3, wherein the orthosensor data is obtained via an orthosensor, the orthosensor comprising a poly insert.

7. The system of claim 1, wherein the medical data includes one or more of narrative textual data, numerical measurements, recorded signals, images, pam scores, and/or demographics.

8. (canceled)

9. The system of claim 1, wherein the osteotomy data is associated with a specific robot-assisted laser osteotomy operation performed on a specific subject.

10. The system of claim 1, wherein the osteotomy data is aggregated from among a plurality of robot-assisted laser osteotomy operations performed on a plurality of subjects.

11. The system of claim 1, wherein the one or more attributes include one or more of a cut quality, a tissue analysis, a hyperspectral result, and/or a laser pulse ablation measurement.

12. (canceled)

13. (canceled)

14. (canceled)

15. (canceled)

16. (canceled)

17. The system of claim 11, wherein differences in laser pulse ablation between subjects are determined based on the one or more attributes.

18. (canceled)

19. (canceled)

20. The system of claim 17, wherein the differences in laser pulse ablation between subjects are a basis for determining a safe set of parameters for an individual subject.

21. The system of claim 17, wherein the differences in laser pulse ablation between subjects are a basis for determining a customized laser dose based on a given subjects own body and conditions.

22. The system of claim 11, wherein data-informed implant designs are designed based on the one or more attributes.

23. (canceled)

24. (canceled)

25. (canceled)

26. (canceled)

27. The system of claim 1, wherein the intraoperative function includes one or more of a fully autonomous surgery function, an implant positioning guidance function, a point of no return warnings function, and/or a time to completion estimates function.

28. (canceled)

29. (canceled)

30. (canceled)

31. (canceled)

32. (canceled)

33. (canceled)

34. The system of claim 1, wherein the post-operative function includes one or more of an operation outcome reports function, a data-informed rehabilitation plans function, a certification via verifiable use statistics function, and/or a collated results function.

35. The system of claim 34, wherein the operation outcome reports function facilitates generation of an operation outcome report with intra-operatively collected data.

36. (canceled)

37. (canceled)

38. (canceled)

39. (canceled)

40. (canceled)

41. A method for robot-assisted laser osteotomy, the method comprising:
receiving osteotomy data obtained in connection with robot-assisted laser osteotomy, the osteotomy data including one or more of device data, medical data, and/or outcome data;
analysing the osteotomy data to determine one or more attributes associated with osteotomy; and
determining, based on the one or more attributes, one or more functions associated with osteotomy, the one or more functions including one or both of an intraoperative function or a post-operative function.

42. (canceled)

43. The method of claim 41, wherein the device data includes one or more of video data, laser data, operating data, hyperspectral data, and/or orthosensor data.

44. The method of claim 43, wherein soft tissue damage is detected and/or measured based on the hyperspectral data.

45. The method of claim 43, wherein bone quality is determined based on the hyperspectral data.

46. The method of claim 43, wherein the orthosensor data is obtained via an orthosensor, the orthosensor comprising a poly insert.

47. The method of claim 41, wherein the medical data includes one or more of narrative textual data, numerical measurements, recorded signals, images, pain scores, and/or demographics.

48. (canceled)

49. The method of claim 41, wherein the osteotomy data is associated with a specific robot-assisted laser osteotomy operation performed on a specific subject.

50. The method of claim 41, wherein the osteotomy data is aggregated from among a plurality of robot-assisted laser osteotomy operations performed on a plurality of subjects.

51. The method of claim 41, wherein the one or more attributes include one or more of a cut quality, a tissue analysis, a hyperspectral result, and/or a laser pulse ablation measurement.

52. (canceled)

53. (canceled)

54. (canceled)

55. (canceled)

56. (canceled)

57. The method of claim 51, wherein differences in laser pulse ablation between subjects are determined based on the one or more attributes.

58. (canceled)

59. (canceled)

60. The method of claim 57, wherein the differences in laser pulse ablation between subjects are a basis for determining a safe set of parameters for an individual subject.

61. The method of claim 57, wherein the differences in laser pulse ablation between subjects are a basis for determining a customized laser dose based on a given subjects own body and conditions.

62. The method of claim 51, wherein data-informed implant designs are designed based on the one or more attributes.

63. (canceled)

64. (canceled)

65. (canceled)

66. (canceled)

67. The method of claim 41, wherein the intraoperative function includes one or more of a fully autonomous surgery function, an implant positioning guidance function, a point of no return warnings function, and/or a time to completion estimates function.

68. (canceled)

69. (canceled)

70. (canceled)

71. (canceled)

72. (canceled)

73. (canceled)

74. The method of claim 41, wherein the post-operative function includes one or more of an operation outcome reports function, a data-informed rehabilitation plans function, a certification via verifiable use statistics function, and/or a collated results function.

75. The method of claim 74, wherein the operation outcome reports function facilitates generation of an operation outcome report with intra-operatively collected data.

76. (canceled)

77. (canceled)

78. (canceled)

79. (canceled)

80. (canceled)

81. A non-transitory computer-readable storage medium having instructions embodied thereon, the instructions being executable by one or more processors to perform a method for robot-assisted laser osteotomy, the method comprising:
receiving osteotomy data obtained in connection with robot-assisted laser osteotomy, the osteotomy data including one or more of device data, medical data, and/or outcome data;
analysing the osteotomy data to determine one or more attributes associated with osteotomy; and
determining, based on the one or more attributes, one or more functions associated with osteotomy, the one or more functions including one or both of an intraoperative function or a post-operative function.

82. (canceled)

83. The computer-readable storage medium of claim 81, wherein the device data includes one or more of video data, laser data, operating data, hyperspectral data, and/or orthosensor data.

84. The computer-readable storage medium of claim 83, wherein soft tissue damage is detected and/or measured based on the hyperspectral data.

85. The computer-readable storage medium of claim 83, wherein bone quality is determined based on the hyperspectral data.

86. The computer-readable storage medium of claim 83, wherein the orthosensor data is obtained via an orthosensor, the orthosensor comprising a poly insert.

87. The computer-readable storage medium of claim 81, wherein the medical data includes one or more of narrative textual data, numerical measurements, recorded signals, images, pain scores, and/or demographics.

88. (canceled)

89. The computer-readable storage medium of claim 81, wherein the osteotomy data is associated with a specific robot-assisted laser osteotomy operation performed on a specific subject.

90. The computer-readable storage medium of claim 81, wherein the osteotomy data is aggregated from among a plurality of robot-assisted laser osteotomy operations performed on a plurality of subjects.

91. The computer-readable storage medium of claim 81, wherein the one or more attributes include one or more of a cut quality, a tissue analysis, a hyperspectral result, and/or a laser pulse ablation measurement.

92. (canceled)

93. (canceled)

94. (canceled)

95. (canceled)

96. (canceled)

97. The computer-readable storage medium of claim 91, wherein differences in laser pulse ablation between subjects are determined based on the one or more attributes.

98. (canceled)

99. (canceled)

100. The computer-readable storage medium of claim 97, wherein the differences in laser pulse ablation between subjects are a basis for determining a safe set of parameters for an individual subject.

101. The computer-readable storage medium of claim 97, wherein the differences in laser pulse ablation between subjects are a basis for determining a customized laser dose based on a given subjects own body and conditions.

102. The computer-readable storage medium of claim 91, wherein data-informed implant designs are designed based on the one or more attributes.

103. (canceled)

104. (canceled)

105. (canceled)

106. (canceled)

107. The computer-readable storage medium of claim 81, wherein the intraoperative function includes one or more of a fully autonomous surgery function, an implant positioning guidance function, a point of no return warnings function, and/or a time to completion estimates function.

108. (canceled)

109. (canceled)

110. (canceled)

111. (canceled)

112. (canceled)

113. (canceled)

114. The computer-readable storage medium of claim 81, wherein the post-operative function includes one or more of an operation outcome reports function, a data-informed rehabilitation plans function, a certification via verifiable use statistics function, and/or a collated results function.

115. The computer-readable storage medium of claim 114, wherein the operation outcome reports function facilitates generation of an operation outcome report with intra-operatively collected data.

116. (canceled)

117. (canceled)

118. (canceled)

119. (canceled)

120. (canceled)
